(19) United States
(12) Patent Application Publication
(10) Pub. No.: US 2010/0047230 A1
(43) Pub. Date: Feb. 25, 2010

(54) ANTI HER2/NEU ANTIBODY

(75) Inventors: Avgi Mamalaki, Athens (GR); Maria Belimezi, Larissa (GR); Danai Papanastassiou, Athens (GR); Efrossini Merkouri, Athens (GR); Konstantinos Baxevanis, Glyfada (GR)

Correspondence Address: BROOKS KUSHMAN P.C., 1000 TOWN CENTER, TWENTY-SECOND FLOOR, SOUTHFIELD, MI, 48075, US

(73) Assignee: HELLENIC PASTEUR INSTITUTE, Athens (GR)

(21) Appl. No.: 11/816,763

(22) PCT Filed: Feb. 20, 2006
(86) PCT No.: PCT/IB2006/000579

§ 371 Date: Nov. 21, 2008

(30) Foreign Application Priority Data
Feb. 21, 2005 (GB) 0503546.4

(51) Int. Cl. A61K 39/395; C07K 16/00; C07H 21/04; C12N 15/63; C12N 5/00; G01N 33/53

(52) U.S. Cl. 424/130.1; 530/387.1; 530/387.3; 530/391.3; 530/391.7; 536/23.53; 435/320.1; 435/325; 435/7.1

(57) ABSTRACT

The present invention describes an isolated antibody or a fragment, variant or derivative thereof, capable of specifically binding to Her2/neu comprising a heavy chain variable domain wherein said heavy chain variable domain comprises an amino acid sequence as set out in at least one of SEQ ID NOs: 1, 2 or 3 or a sequence having at least 75% identity (homology) thereto or a functional fragment thereof as well as nucleic acid sequences encoding the same and methods for their production. The invention also relates to a method for the treatment of Her2/neu expressing cancer in a patient comprising administering to the patient in need of such treatment a therapeutically effective amount of any of those antibody, polypeptide or nucleic acid molecules described herein.

The present invention relates to antibodies that recognise Her2/neu. In particular, the present invention relates to particular antibodies which exert an antiproliferative effect on Her2/neu expressing cells.

### BACKGROUND TO THE INVENTION

The Her2/neu (ErbB2) (also referred to herein as Her2) gene encodes a Mᵣ 185,000 transmembrane glycoprotein that belongs to the erbB family of epidermal growth factor receptors (Akiyama et al., 1986; Rubin and Yarden, 2001). Ligand binding induces the formation of erbB homo- and heterodimers, resulting in activation of the cytoplasmic kinase domain (Marmor et al., 2004). Her2/neu is a ligand-less receptor and is the preferred heterodimerization partner among ligand bound EGFR family Her1 (EGFR), Her3 and Her4 (Yarden and Sliwkowski, 2001). As, a co-receptor, Her2/neu mediates signal transduction, resulting in mitogenesis, apoptosis, angiogenesis and cell differentiation (Harari and Yarden, 2000; Tzahar et al., 1997). Any alteration of the tightly regulated erbB receptor signaling pathways result in major abnormalities and tumourigenesis.

The Her2/neu gene is amplified and overexpressed in ˜20-30% of invasive breast carcinomas and is associated with increased metastatic potential and poor prognosis (Press et al., 1993; Ross and Fletcher, 1998; Ross et al., 2003; Slamon, 1987). Furthermore, the overexpression of Her2/neu receptor occurs in various human cancers, including ovary, prostate, gastric, lung, bladder and kidney carcinomas (Koeppen et al., 2001; Slamon, 1987). Her2/neu is able to exert its effect through homodimers, following a ligand-independent mode of activation in the high-expressing Her2/neu cells. The Her2/neu receptor undergoes slow proteolytic cleavage and the resulting Mᵣ 110,000 receptor extracellular domain (ECD) can be detected in the conditioned medium (Zabrecky et al., 1991) and in the serum of breast cancer patients (Molina et al., 2001, 2002). Proteolytic cleavage also generates a Mᵣ 95,000 amino-terminally truncated membrane-associated fragment with in vitro kinase activity (Christianson et al., 1998). On the other hand, Her2/neu can be activated by ligand-mediated stimulation of another erbB receptor. In low expressing Her2/neu cells signal transduction is enhanced significantly through Her2/neu heterodimers (Citri et al., 2003; Holbro et al., 2003; Mellinghoff et al., 2004).

Recent crystallographic studies of the extracellular region of Her2/neu revealed a fixed conformation that resembles a ligand-activated state and showed that Her2/neu poised to interact with other erbB receptors in the absence of direct ligand binding (Cho et al., 2003; Garrett et al., 2003). It is proposed that once the ECD is removed, the remnant Her2/neu transmembrane and intracellular domains are thought to self-associate and to interact with uncleaved Her2/neu, resulting in constitutive kinase activation (Burgess et al., 2003; Molina et al., 2001).

Blocking the Her2/neu signaling and limiting the number of available membrane molecules, has been the focus of most therapeutic approaches. Herceptin, is a humanized version of murine 4D5 mAb, that binds to the juxtamembrane region of Her2/neu (Cho et al., 2003) and exerts its antiproliferative activity by cleavage prevention of the receptor's extracellular domain (which leads to receptor constitutive activation) and receptor downmodulation (Wang et al., 2004).

mAb 2C4, that binds to a different epitope than Herceptin in the receptor's extracellular domain near the center of domain II (Franklin et al., 2004), sterically blocks the association of HER2 with other erbB family members and disrupts ligand activation (Agus et al., 2002). Pertuzumab, the humanized 2C4 mAb is recently introduced in clinical trials targeting low expressing Her2/neu cancers (Franklin et al., 2004). Moreover, Trastuzumab and Pertuzumab synergistically inhibit the survival of Her2/neu overexpressing breast cancer cell lines (Nahta et al., 2004).

An internalized human scFv displayed on phage (Erbicin) was isolated with cytostatic/cytotoxic effect on HER2/neu positive cell lines (De Lorenzo et al., 2002). As an antitumor agent, the soluble form of this scFv was less active than the phage format; the soluble form probably has a conformation with lower stability. Recently, from this scFv a compact reduced version of an IgG was produced as a novel antitumor agent with antiproliferative effect on tumor target cells that can induce antibody-dependent cellular cytotoxicity (ADCC) and complement-dependent cytotoxicity (CDC) (De Lorenzo et al., 2004).

Herceptin is the first antibody to enter the clinic for treatment of Her2/neu positive cancers but only has antitumor activity in up to 30% of patients overexpressing Her2/neu and does not have any activity against tumors that express lower levels of Her2/neu (Camirand et al., 2002; Lu et al., 2001).

Accordingly, there is a need for alternative antibodies directed to Her2/neu that show improved therapeutic potential.

### STATEMENT OF INVENTION

The present invention relates to the identification of improved antibodies that recognise the extracellular domain (ECD) of Her2/neu receptor. The antibodies of the invention and, in particular the human Fab fragment, referred to herein as Fab63, compete with Herceptin in binding to soluble Her2/neu receptor and can bind to the native receptor in the surface of Her2/neu overexpressing cells. Furthermore, the antibodies of the invention exemplified by Fab63 have significant antiproliferative effects in SKBR3 and MDA-MB-453 cancer cells where ligand-independent mechanisms dominate signal induction. Moreover, in the presence of the ligand HRG-β1, growth inhibition was detected in high and low Her2/neu expressing cells MDA-MB-453 and MCF7. Unlike Herceptin, Fab63 is strongly internalized. In addition, compared to Erbicin the time of internalization of Fab63 is significantly reduced in high expressing Her2/neu cells. This combination of properties makes the antibodies of the invention suitable anti-cancer agents for diagnosis and therapeutics.

Accordingly, in a first aspect of the invention there is provided an isolated antibody or a fragment, variant or derivative thereof, capable of specifically binding to Her2/neu comprising a heavy chain variable domain wherein said heavy chain variable domain comprises an amino acid sequence as set out in at least one of SEQ ID NOs: 1, 2 or 3 or a sequence having at least 75% identity (homology) thereto or a functional fragment thereof.

In a preferred embodiment, said isolated antibody in accordance with the first aspect further comprises a light chain variable domain wherein said light chain variable domain comprises an amino acid sequence as set out in at least one of SEQ ID NOs: 4, 5 or 6 or a sequence having at least 75% identity (homology) thereto or a functional fragment thereof.

Suitably, the antibody comprises at least one of the amino acid sequences shown in any of SEQ ID NOs: 1 to 6 or a sequence having at least 75% identity (homology) thereto or a functional fragment thereof.

Preferably, the homologous amino acid sequence includes an amino acid sequence which may be at least 75, 80, 81, 85 or 90% identical, preferably at least 95, 96, 97, 98 or 99% identical to the sequence.

In another embodiment, said antibody in accordance with the invention comprises a combination of at least two of SEQ ID NOs: 1 to 6. Preferably, said antibody comprises a heavy chain variable domain comprising a combination of all of SEQ ID NOs: 1 to 3. In another embodiment, said antibody comprises a light chain variable domain comprising a combination of all of SEQ ID NOs: 4 to 6.

In a preferred embodiment the antibody of the invention comprises a heavy chain variable domain having an amino acid sequence as set out in SEQ ID NO: 7. In another embodiment, the antibody comprises a light chain variable domain having an amino acid sequence as set out in SEQ ID NO: 8.

In yet another embodiment, the antibody comprises a heavy chain variable domain having an amino acid sequence as set out in SEQ ID NO: 7 in combination with a light chain variable domain having an amino acid sequence as set out in SEQ ID NO: 8. According to the above aspects of the invention, the term “specific binding to Her2/neu” means that within a mixture of reagents comprising Her2/neu in addition to other alternative antigens, only Her2/neu is bound. That is, the binding of an anti-Her2/neu antibody according to the present invention is selective for Her2. One method of showing specific binding of an antibody in accordance with the invention is a competitive binding assay with other known anti-Her2/neu antibodies such as Herceptin wherein an ability to displace or compete with Herceptin binding is indicative of specific Her2/neu binding. Suitable methods are described herein. Alternatively affinity measurements may be made using methods known to those skilled in the art including using BIACORE measurements as herein described. Other suitable methods for determining specific binding will be familiar to those skilled in the art.

Preferably, an isolated antibody in accordance with the invention is capable of rapid internalisation.

By “internalisation” is meant that the antibody is taken into the cell on which Her2/neu is expressed. Suitably, the antibody is internalised rapidly i.e. internalisation is observed preferably within approximately 30 mins of incubation of the antibody with the cell expressing the Her2/neu receptor at 37° C. and, suitably, a maximum efficiency of internalisation is observed after 2 hours of incubation at 37° C.

Suitably, the isolated antibody in accordance with the invention is able to exert an antiproliferative effect when provided to a cell expressing Her2.

According to the above aspects of the invention, the term “exerting an anti-proliferative effect” means that inhibiting cell growth in a sample treated with an antibody when compared to an untreated sample and/or with an unrelated antibody. Suitable assays for determining cell growth are known to those skilled in the art and described herein. Preferably an antiproliferative effective is detected by measuring a % inhibition of cell growth. Suitably, 10%, 20%, 30%, 40% or 50% growth inhibition is detected and indicative of an antiproliferative effect of an antibody in accordance with the invention.

Suitably the antibody is an Fv fragment comprising a CDR as set out in any of SEQ ID NOs: 1 to 6 and framework regions. In another embodiment, the antibody is a Fab fragment. Suitably, said Fab fragment comprises constant domains of the heavy and light chains. In yet another embodiment, the antibody is a scFv fragment. Suitably, in this embodiment, said heavy chain variable domain and said light chain variable domain are operably linked to form a scFv.

The small size of Fab and scFv fragments can be advantageous over whole or compact antibodies in penetrating solid tumors and targeting malignant cells especially when they employed with a cytotoxic or cytostatic load (Harris, 2004). Internalized Fabs and scFvs have also been isolated (Poul et al., 2000) and are of great interest since they are more potent tools for delivering immunotoxins, immunoliposomes or DNA into cells (Fominaya and Wels, 1996; Park et al., 2002; Wang et al., 2001), by going beyond the membrane barrier and enter the cytoplasm, once bound in the receptor. In addition, internalized antibodies have limited cytotoxicity compared to antibodies that are constantly exposed in the membrane surface.

In another embodiment, the antibody may be oligomerized into mono- or bi-specific diabodies, or even into higher order valency antibody fragments.

In a particularly preferred embodiment the Fab fragment is Fab63 as described herein.

Advantageously, Fab63 is derived from a human library and is non-immunogenic if administered to humans.

Advances in antibody engineering allowed the isolation of fully human antibody Fab and scFv fragments from phage display human antibody libraries (Carter, 2001). The ability to generate fully human antibody fragments is important because it overcame the host anti-mouse antibody (HAMA) response produced by antibody chimerization or humanization (Brekke and Sandlie, 2003). Accordingly, in a preferred embodiment, the antibody in accordance with the invention is human.

In another embodiment, the antibody may be modified so as to increase stability and/or half-life. For example, half-life of an antibody may be increased by pegylation of the antibody or antibody fragment (see, for example, Chapman A P, Adv Drug Deliv Rev 2002, 54; 531-545). The antibody in accordance with the invention may also be generated as an armed molecule adopting enhancement manipulation techniques. In addition, the whole antibody may be constructed, for example by adding the Fc constant part of the human immunoglobulin, or by generating mutated antibodies using techniques such as chain shuffling, as described herein.

The antibody may be generated as an immunoconjugate comprising an antibody component linked to a diagnostic or therapeutic agent. Said linkage may be by means recognised by those skilled in the art including chemical conjugation or genetic fusion.

Said therapeutic agent may be selected from the group consisting of an antibody, an immunomodulator, a hormone, a cytotoxic agent, a drug, a toxin, an enzyme, a radionuclide, antisense oligonucleotide or combination thereof.

Accordingly, in a another embodiment, there is provided an antibody in accordance with the invention conjugated to a second molecule wherein the second molecule is selected from the group consisting of a cytotoxic drug, cytostatic drug, immunotoxin, immunoliposome, DNA molecule.

In another aspect of the invention, there is provided an isolated amino acid sequence as set out in any of SEQ ID NOs: 1 to 8 or a sequence having at least 75% identity (homology) thereto or a functional fragment thereof.

Suitably, there is provided a CDR sequence comprising an amino acid sequence as set out in any of SEQ ID NOs: 1 to 6.

In another aspect of the invention there is provided an isolated nucleic acid molecule encoding any one or more antibody molecules and/or CDR or amino acid sequences in accordance with any embodiment of the aspects of the invention, or a homologue thereof.

Suitably said isolated and/or purified nucleic acid molecule encodes an antibody comprising the amino acid sequence as shown in any of SEQ ID NOs: 1 to 8 or a sequence having at least 75% identity (homology) thereto or an effective fragment thereof. In another embodiment, the invention provides an isolated and/or purified nucleic acid molecule comprising a nucleotide sequence that is the same as, or is complementary to, or contains any suitable codon substitutions for any of those of any of SEQ ID NOs: 1 to 8 or comprises a sequence which has at least 75%, 80%, 85%, 90%, 95%, 96%, 97%, 98% or 99% sequence homology with any of SEQ ID NOs: 1 to 8.

In one embodiment, there is provided a nucleic acid molecule comprising a sequence as set out in any of SEQ ID NOs: 9 to 16.

In another aspect, the invention provides a plasmid or vector system comprising a nucleic acid encoding an antibody as described herein or a homologue or derivative thereof. Preferably, the plasmid or vector system comprises a nucleic acid sequence as set out in any of SEQ ID Nos: 9 to 16 or a sequence that is at least 75% homologous thereto or an effective fragment thereof. Suitably the plasmid or vector system is an expression vector for the expression of any of the antibodies encoded by a nucleic acid sequence as set out in any of SEQ ID Nos: 9 to 16 or a sequence that is at least 75% homologous (identical) thereto in a microorganism. Suitable expression vectors are described herein.

In another aspect of the invention there is provided a host cell transformed or transfected with a nucleic acid encoding an antibody as described herein.

Suitably, the host cell in accordance with this aspect of the invention comprises a antibody which comprises an amino acid sequence, or functional fragment thereof, as set out in any of SEQ ID NOs: 1 to 8 or a sequence that is at least 75% homologous thereto.

In one embodiment, said host cell is transformed with a vector in accordance with the invention.

Preferably said host cell produces an antibody.

Suitably the host cell is derived from a microorganism including bacteria and fungi, including yeast. In a particularly preferred embodiment the host cell is a prokaryotic bacterial cell. Suitable bacterial host cells include bacteria from different prokaryotic taxonomic groups including proteobacteria, including members of the alpha, beta, gamma, delta and epsilon subdivision, gram-positive bacteria such as Actinomycetes, Firmicutes, Clostridium and relatives, flavobacteria, cyanobacteria, green sulfur bacteria, green non-sulfur bacteria, and archaea. Particularly preferred are the Enterobacteriaceae such as Escherichia coli proteobacteria belonging to the gamma subdivision and low GC-Gram positive bacteria such as Bacillus.

Suitable fungal host cells include yeast selected from the group consisting of Ascomycota including Saccharomycetes such as Pichia, Hansenula, and Saccharomyces, Schizosaccharmycetes such as Schizosaccharomyces pombe and anamorphic Ascomycota including Aspergillus.

The host cell may also be a eukaryotic cell. Suitable eukaryotic host cells will be familiar to those skilled in the art and include mammalian, plant and protozoa cells (such as Leishmania etc.).

Other suitable eukaryotic host cells include insect cells such as SF9, SF21, Trychplusiani and M121 cells. For example, the antibodies according to the invention can advantageously be expressed in insect cell systems. As well as expression in insect cells in culture, phytase genes can be expressed in whole insect organisms. Virus vectors such as baculovirus allow infection of entire insects. Large insects, such as silk moths, provide a high yield of heterologous protein. The protein can be extracted from the insects according to conventional extraction techniques. Expression vectors suitable for use in the invention include all vectors which are capable of expressing foreign proteins in insect cell lines.

In another aspect, there is provided a method for synthesising an antibody molecule in accordance with the invention.

According to the above aspects of the invention, the term ‘synthesising the antibody’ includes within its scope the selection of whole/intact antibodies comprising the sequences referred to above, and/or the selection of antibody fragments comprising the sequences referred to above and their subsequent assembly. Furthermore, the term includes within its scope mutating suitable sequences at the amino acid level or nucleic acid level, in order to generate the sequences referred to above. Mutation may take the form of a substitution, deletion, inversion or insertion. Advantageously the mutation will be a substitution. Methods for performing mutagenesis and manipulation of nucleic acid or amino acid sequences involve standard laboratory techniques and will be familiar to those skilled in the art.

In addition the term ‘synthesising the antibody’ includes within its scope assembling de novo or synthesising de novo a nucleic acid construct encoding the various sequences or fragments thereof, referred to above. The synthesis of nucleic acid may include a PCR based approach. Those skilled in the art will be aware of other suitable methods for the synthesis of nucleic acid encoding the sequences referred to above.

The antibodies and/or nucleic acid constructs encoding them have significant therapeutic value.

The antibodies of the present invention are of particular use for in vivo prophylactic and therapeutic purposes. In particular, the present inventors have found that particular antibodies according to the invention are capable of inhibiting cell growth of a Her2/neu expressing cell line.

Accordingly, in another aspect of the invention there is provided a composition comprising any of those molecules selected from the group consisting of the following: an antibody molecule according to the invention, one or more CDRs of the invention and a nucleic acid construct according to the invention and a pharmaceutically acceptable carrier, diluent or excipient.

In another aspect, there is provided a method for the treatment of Her2/neu expressing cancer in a patient comprising administering to the patient in need of such treatment a therapeutically effective amount of one or more antibody molecules in accordance with the invention.

In addition, said method of treatment may comprise administering a nucleic acid molecule in accordance with the invention.

In a further aspect, there is provided the use of an antibody in accordance with the invention in the preparation of a medicament for use in the treatment of cancers expressing Her2.

Additionally, there is provided the use of a nucleic acid sequence in accordance with the invention in the preparation of a medicament for use in the treatment of cancers expressing Her2.

In another aspect, there is provided a method for diagnosing or treating a cancer expressing Her2/neu comprising contacting a sample with an antibody as described herein.

In a further aspect, there is provided the use of an antibody in accordance with the invention in diagnosis.

In another aspect of the invention, there is provided a construct for expressing the extracellular domain of Her2/neu (ECD) in P. pastoris. Suitably said construct is as set out in the Examples section herein. In a yet further aspect, there is provided the use of such a construct in a method for identifying antibodies.

Furthermore, the present invention provides the use an ECD expressed in P. pastoris in a vaccine. Suitably said ECD is as described in the Examples section herein.

### DETAILED DESCRIPTION

### Definitions

Immunoglobulins molecules, according to the present invention, refer to any moieties which are capable of binding to a target. In particular, they include members of the immunoglobulin superfamily, a family of polypeptides which comprise the immunoglobulin fold characteristic of antibody molecules, which contains two beta sheets and, usually, a conserved disulphide bond. Members of the immunoglobulin superfamily are involved in many aspects of cellular and non-cellular interactions in vivo, including widespread roles in the immune system (for example, antibodies, T-cell receptor molecules and the like), involvement in cell adhesion (for example the ICAM molecules) and intracellular signalling (for example, receptor molecules, such as the PDGF receptor).

Antibodies as used herein, refers to complete antibodies or antibody fragments capable of binding to a selected target, and including Fv, single chain antibodies (scFv), F(ab′) and F(ab′)₂, monoclonal and polyclonal antibodies, engineered antibodies including chimeric, CDR-grafted and humanised antibodies, and artificially selected antibodies produced using phage display or alternative techniques. Suitably, the antibodies and fragments thereof may be humanised antibodies. Small fragments, such as Fv and scFv, possess advantageous properties for diagnostic and therapeutic applications on account of their small size and consequent superior tissue distribution. Fusion proteins and other synthetic proteins which comprise the antigen-binding site of the antibody are also included.

Techniques for the production of Fab fragments are described for example in U.S. Pat. No. 5,658,727. Techniques for the production of single chain antibodies are described for example in U.S. Pat. No. 4,946,778.

As herein defined the term ‘antibody’ includes within its scope molecules which comprise an antigen binding moiety comprising a heavy chain variable domain or a light chain variable domain alone. In a preferred embodiment of the invention, an antibody comprises a heavy chain variable domain only.

Antibodies of the invention may also be modified. The term “modified antibody” is also intended to include antibodies, such as monoclonal antibodies, chimeric antibodies, and humanized antibodies which have been modified by, e.g., deleting, adding, or substituting portions of the antibody. For example, an antibody can be modified by deleting the constant region and replacing it with a constant region meant to increase half-life, e.g., serum half-life, stability or affinity of the antibody. A “modified antibody” in accordance with the invention may also be modified by “chain shuffling” technology in order to obtain better affinities and/or to identify the specific amino acids that are mutated by an antigen driven mechanism (see, for example, Park et al. 2000. Affinity maturation of natural antibody using a chain shuffling technique and the expression of recombinant antibodies in Escherichia coli. Biochem Biophys Res Commun 275:553; Huls et al. 2001. Tumor cell killing by in vitro affinity-matured recombinant human monoclonal antibodies. Cancer Immunol Immunother 50:163; Fostieri et al., 2005. Isolation of potent human Fab fragments against a novel highly immunogenic region on human muscle acetylcholine receptor which protect the receptor from myasthenic autoantibodies. Eur J Immunol. 35:632).

Heavy chain variable domain refers to that part of the heavy chain of an immunoglobulin molecule which forms part of the antigen binding site of that molecule. The VH4 subgroup describes a particular sub-group of heavy chain variable regions (the VH4). Generally immunoglobulin molecules having a variable chain amino acid sequence falling within this group possess a VH amino acid sequence which can be described by the VH4 consensus sequence in the Kabat database.

Light-chain variable domain refers to that part of the light chain of an immunoglobulin molecule which forms part of the antigen binding site of that molecule. The VK subgroup of immunoglobulin molecules describes a particular sub-group of variable light chains. Generally immunoglobulin molecules having a variable chain amino acid sequence falling within this group possess a VL amino acid sequence which can be described by the VK consensus sequence in the Kabat database.

Framework region of an immunoglobulin heavy and light chain variable domain. The variable domain of an immunoglobulin molecule has a particular 3 dimensional conformation characterised by the presence of an immunolgobulin fold. Certain amino acid residues present in the variable domain are responsible for maintaining this characteristic immunoglobulin domain core structure. These residues are known as framework residues and tend to be highly conserved.

CDR (complementarity determining region) of an immunoglobulin molecule heavy and light chain variable domain describes those amino acid residues which are not framework region residues and which are contained within the hypervariable loops of the variable regions. These hypervariable loops are directly involved with the interaction of the immunoglobulin with the ligand. Residues within these loops tend to show less degree of conservation than those in the framework region.

Consensus sequence of V_{H} and V_{L} chains in the context of the present invention refers to the consensus sequences of those V_{H} and V_{L} chains from immunoglobulin molecules which can bind selectively to a ligand. The residue which is most common in any one given position, when the sequences of those immunoglobulins which can bind are compared is chosen as the consensus residue for that position.

Specific (antibody) binding in the context of the present invention, means that the interaction between the antibody and the ligand are selective, that is, in the event that a number of molecules are presented to the antibody, the latter will only bind to one or a few of those molecules presented. Advantageously, the antibody-ligand interaction will be of high affinity. The interaction between immunoglobulin and ligand will be mediated by non-covalent interactions such as hydrogen bonding and Van der Waals forces.

### Isolated

In one aspect, preferably the nucleotide or amino acid sequence is in an isolated form. The term “isolated” means that the sequence is at least substantially free from at least one other component with which the sequence is naturally associated in nature and as found in nature.

### Purified

In one aspect, preferably the nucleotide or amino acid sequence is in a purified form. The term “purified” means that the sequence is in a relatively pure state—e.g. at least about 90% pure, or at least about 95% pure or at least about 98% pure.

### Nucleic Acid Molecule or Nucleotide Sequence

The scope of the present invention encompasses nucleotide sequences encoding antibodies having the specific properties as defined herein.

The term “nucleic acid molecule” or “nucleotide sequence” as used herein refers to an oligonucleotide sequence, nucleic acid or polynucleotide sequence, and variant, homologues, fragments and derivatives thereof (such as portions thereof). The nucleotide sequence may be of genomic or synthetic or recombinant origin, which may be double-stranded or single-stranded whether representing the sense or anti-sense strand.

The term “nucleotide sequence” or “nucleic acid molecule” in relation to the present invention includes genomic DNA, cDNA, synthetic DNA, and RNA. Preferably it means DNA, more preferably cDNA sequence coding for the present invention.

In a preferred embodiment, the nucleotide sequence when relating to and when encompassed by the per se scope of the present invention does not include the native nucleotide sequence according to the present invention when in its natural environment and when it is linked to its naturally associated sequence(s) that is/are also in its/their natural environment. For ease of reference, we shall call this preferred embodiment the “non-native nucleotide sequence”. In this regard, the term “native nucleotide sequence” means an entire nucleotide sequence that is in its native environment and when operatively linked to an entire promoter with which it is naturally associated, which promoter is also in its native environment. However, the amino acid sequence encompassed by scope the present invention can be isolated and/or purified post expression of a nucleotide sequence in its native organism. Preferably, however, the amino acid sequence encompassed by scope of the present invention may be expressed by a nucleotide sequence in its native organism but wherein the nucleotide sequence is not under the control of the promoter with which it is naturally associated within that organism.

### Preparation of a Nucleotide Sequence

Typically, the nucleotide sequence encompassed by scope of the present invention or the nucleotide sequences for use in the present invention are prepared using recombinant DNA techniques (i.e. recombinant DNA). However, in an alternative embodiment of the invention, the nucleotide sequence could be synthesised, in whole or in part, using chemical methods well known in the art (see Caruthers M H et al., (1980) Nuc Acids Res Symp Ser 215-23 and Horn T et al., (1980) Nuc Acids Res Symp Ser 225-232).

A nucleotide sequence encoding either an antibody which has the specific properties as defined herein or an antibody which is suitable for modification may be identified and/or isolated and/or purified from any phage, cell or organism producing said antibody. Various methods are well known within the art for the identification and/or isolation and/or purification of nucleotide sequences. By way of example, PCR amplification techniques to prepare more of a sequence may be used once a suitable sequence has been identified and/or isolated and/or purified.

By way of further example, a genomic DNA and/or cDNA library may be constructed using chromosomal DNA or messenger RNA from the cell producing the antibody.

In a yet further alternative, the nucleotide sequence encoding the antibody may be prepared synthetically by established standard methods, e.g. the phosphoroamidite method described by Beucage S. L. et al., (1981) Tetrahedron Letters 22, p 1859-1869, or the method described by Matthes et al., (1984) EMBO J. 3, p 801-805. In the phosphoroamidite method, oligonucleotides are synthesised, e.g. in an automatic DNA synthesiser, purified, annealed, ligated and cloned in appropriate vectors.

The nucleotide sequence may be of mixed genomic and synthetic origin, mixed synthetic and cDNA origin, or mixed genomic and cDNA origin, prepared by ligating fragments of synthetic, genomic or cDNA origin (as appropriate) in accordance with standard techniques. Each ligated fragment corresponds to various parts of the entire nucleotide sequence. The DNA sequence may also be prepared by polymerase chain reaction (PCR) using specific primers, for instance as described in U.S. Pat. No. 4,683,202 or in Saiki R K et al., (Science (1988) 239, pp 487-491).

Due to degeneracy in the genetic code, nucleotide sequences may be readily produced in which the triplet codon usage, for some or all of the amino acids encoded by the original nucleotide sequence, has been changed thereby producing a nucleotide sequence with low homology to the original nucleotide sequence but which encodes the same, or a variant, amino acid sequence as encoded by the original nucleotide sequence. For example, for most amino acids the degeneracy of the genetic code is at the third position in the triplet codon (wobble position) (for reference see Stryer, Lubert, Biochemistry, Third Edition, Freeman Press, ISBN 0-7167-1920-7) therefore, a nucleotide sequence in which all triplet codons have been “wobbled” in the third position would be about 66% identical to the original nucleotide sequence however, the amended nucleotide sequence would encode for the same, or a variant, primary amino acid sequence as the original nucleotide sequence.

Therefore, the present invention further relates to any nucleotide sequence that has alternative triplet codon usage for at least one amino acid encoding triplet codon, but which encodes the same, or a variant, polypeptide sequence as the polypeptide sequence encoded by the original nucleotide sequence.

Furthermore, specific organisms typically have a bias as to which triplet codons are used to encode amino acids. Preferred codon usage tables are widely available, and can be used to prepare codon optimised genes. Such codon optimisation techniques are routinely used to optimise expression of transgenes in a heterologous host.

### Amino Acid Sequences

The scope of the present invention also encompasses amino acid sequences of antibodies having the specific properties as defined herein.

As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “antibody”.

The amino acid sequence may be prepared/isolated from a suitable source, or it may be made synthetically or it may be prepared by use of recombinant DNA techniques.

The antibody encompassed in the present invention may be used in conjunction with other antibodies. Thus the present invention also covers a combination of antibodies wherein the combination comprises the antibody of the present invention and another antibody, which may be another antibody according to the present invention. This aspect is discussed in a later section.

Preferably the amino acid sequence when relating to and when encompassed by the per se scope of the present invention is not a native antibody. In this regard, the term “native antibody” means an entire antibody that is in its native environment and when it has been expressed by its native nucleotide sequence.

### Variants/Homologues/Derivatives

The present invention also encompasses the use of variants, homologues and derivatives of any amino acid sequence of an antibody or of any nucleotide sequence encoding such an antibody.

Here, the term “homologue” means an entity having a certain homology with the amino acid sequences and the nucleotide sequences. Here, the term “homology” can be equated with “identity”.

In the present context, a homologous amino acid sequence is taken to include an amino acid sequence which may be at least 75, 80, 81, 85 or 90% identical, preferably at least 95, 96, 97, 98 or 99% identical to the sequence. Typically, the homologues will comprise the same active sites etc.—e.g as the subject amino acid sequence. Although homology can also be considered in terms of similarity (i.e. amino acid residues having similar chemical properties/functions), in the context of the present invention it is preferred to express homology in terms of sequence identity.

By “functional fragment” is meant a fragment of the polypeptide that retains that characteristic properties of that polypeptide. In the context of the present invention, a functional fragment of an antibody as described herein is a fragment that retains Her2/neu binding capability.

In the present context, an homologous nucleotide sequence is taken to include a nucleotide sequence which may be at least 75, 80, 81, 85 or 90% identical, preferably at least 95, 96, 97, 98 or 99% identical to a nucleotide sequence encoding an antibody of the present invention (the subject sequence). Typically, the homologues will comprise the same sequences that code for the same CDRs as the subject sequence. Although homology can also be considered in terms of similarity (i.e. amino acid residues having similar chemical properties/functions), in the context of the present invention it is preferred to express homology in terms of sequence identity.

For the amino acid sequences and the nucleotide sequences, homology comparisons can be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (Devereux et al 1984 Nuc. Acids Research 12 p 387). Examples of other software than can perform sequence comparisons include, but are not limited to, the BLAST package (see Ausubel et al., 1999 Short Protocols in Molecular Biology, 4^{th} Ed—Chapter 18), FASTA (Altschul et al., 1990 J. Mol. Biol. 403-410) and the GENEWORKS suite of comparison tools. Both BLAST and FASTA are available for offline and online searching (see Ausubel et al., 1999, Short Protocols in Molecular Biology, pages 7-58 to 7-60).

However, for some applications, it is preferred to use the GCG Bestfit program. A new tool, called BLAST 2 Sequences is also available for comparing protein and nucleotide sequence (see FEMS Microbiol Lett 1999 174(2): 247-50; FEMS Microbiol Lett 1999 177(1): 187-8 and tatiana@ncbi.nlm.nih.gov).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). For some applications, it is preferred to use the public default values for the GCG package, or in the case of other software, the default matrix, such as BLOSUM62.

Alternatively, percentage homologies may be calculated using the multiple alignment feature in DNASIS™ (Hitachi Software), based on an algorithm, analogous to CLUSTAL (Higgins DG & Sharp PM (1988), Gene 73(1), 237-244).

Once the software has produced an optimal alignment, it is possible to calculate % homology, preferably % sequence identity. The software typically does this as part of the sequence comparison and generates a numerical result.

The sequences may also have deletions, insertions or substitutions of amino acid residues which produce a silent change and result in a functionally equivalent substance. Deliberate amino acid substitutions may be made on the basis of similarity in amino acid properties (such as polarity, charge, solubility, hydrophobicity, hydrophilicity, and/or the amphipathic nature of the residues) and it is therefore useful to group amino acids together in functional groups. Amino acids can be grouped together based on the properties of their side chain alone. However it is more useful to include mutation data as well. The sets of amino acids thus derived are likely to be conserved for structural reasons. These sets can be described in the form of a Venn diagram (Livingstone C. D. and Barton G. J. (1993) “Protein sequence alignments: a strategy for the hierarchical analysis of residue conservation” Comput. Appl Biosci. 9: 745-756) (Taylor W. R. (1986) “The classification of amino acid conservation” J. Theor. Biol. 119; 205-218). Conservative substitutions may be made, for example according to the table below which describes a generally accepted Venn diagram grouping of amino acids.

| SET | | SUB-SET | |
|---|---|---|---|
| Hydrophobic | F W Y H K M I L V A G C | Aromatic | F W Y H |
| | | Aliphatic | I L V |
| Polar | W Y H K R E D C S T N Q | Charged | H K R E D |
| | | Positively | H K R |
| | | charged | |
| | | Negatively | E D |
| | | charged | |
| Small | V C A G S P T N D | Tiny | A G S |

The present invention also encompasses homologous substitution (substitution and replacement are both used herein to mean the interchange of an existing amino acid residue, with an alternative residue) that may occur i.e. like-for-like substitution such as basic for basic, acidic for acidic, polar for polar etc. Non-homologous substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine.

Replacements may also be made by unnatural amino acids.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involves the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al., PNAS (1992) 89(20), 9367-9371 and Horwell D C, Trends Biotechnol. (1995) 13(4), 132-134.

The nucleotide sequences for use in the present invention may include within them synthetic or modified nucleotides. A number of different types of modification to oligonucleotides are known in the art. These include methylphosphonate and phosphorothioate backbones and/or the addition of acridine or polylysine chains at the 3′ and/or 5′ ends of the molecule. For the purposes of the present invention, it is to be understood that the nucleotide sequences described herein may be modified by any method available in the art. Such modifications may be carried out in order to enhance the in vivo activity or life span of nucleotide sequences of the present invention.

The present invention also encompasses the use of nucleotide sequences that are complementary to the sequences presented herein, or any derivative, fragment or derivative thereof. If the sequence is complementary to a fragment thereof then that sequence can be used as a probe to identify similar coding sequences in other organisms etc.

Polynucleotides which are not 100% homologous to the sequences of the present invention but fall within the scope of the invention can be obtained in a number of ways. Other variants of the sequences described herein may be obtained for example by probing DNA libraries made from a range of individuals, for example individuals from different populations. In addition, other homologues may be obtained and such homologues and fragments thereof in general will be capable of selectively hybridising to the sequences shown in the sequence listing herein. Such sequences may be obtained by probing cDNA libraries made from or genomic DNA libraries from other species, and probing such libraries with probes comprising all or part of any one of the sequences in the attached sequence listings under conditions of medium to high stringency. Similar considerations apply to obtaining species homologues and allelic variants of the polypeptide or nucleotide sequences of the invention.

Variants and strain/species homologues may also be obtained using degenerate PCR which will use primers designed to target sequences within the variants and homologues encoding conserved amino acid sequences within the sequences of the present invention. Conserved sequences can be predicted, for example, by aligning the amino acid sequences from several variants/homologues. Sequence alignments can be performed using computer software known in the art. For example the GCG Wisconsin PileUp program is widely used.

The primers used in degenerate PCR will contain one or more degenerate positions and will be used at stringency conditions lower than those used for cloning sequences with single sequence primers against known sequences.

Alternatively, such polynucleotides may be obtained by site directed mutagenesis of characterised sequences. This may be useful where for example silent codon sequence changes are required to optimise codon preferences for a particular host cell in which the polynucleotide sequences are being expressed. Other sequence changes may be desired in order to introduce restriction antibody recognition sites, or to alter the property or function of the polypeptides encoded by the polynucleotides.

Polynucleotides (nucleotide sequences) of the invention may be used to produce a primer, e.g. a PCR primer, a primer for an alternative amplification reaction, a probe e.g. labelled with a revealing label by conventional means using radioactive or non-radioactive labels, or the polynucleotides may be cloned into vectors. Such primers, probes and other fragments will be at least 15, preferably at least 20, for example at least 25, 30 or 40 nucleotides in length, and are also encompassed by the term polynucleotides of the invention as used herein.

Polynucleotides such as DNA polynucleotides and probes according to the invention may be produced recombinantly, synthetically, or by any means available to those of skill in the art. They may also be cloned by standard techniques.

In general, primers will be produced by synthetic means, involving a stepwise manufacture of the desired nucleic acid sequence one nucleotide at a time. Techniques for accomplishing this using automated techniques are readily available in the art.

Longer polynucleotides will generally be produced using recombinant means, for example using a PCR (polymerase chain reaction) cloning techniques. The primers may be designed to contain suitable restriction antibody recognition sites so that the amplified DNA can be cloned into a suitable cloning vector.

### Hybridisation

The present invention also encompasses sequences that are complementary to the nucleic acid sequences of the present invention or sequences that are capable of hybridising either to the sequences of the present invention or to sequences that are complementary thereto.

The term “hybridisation” as used herein shall include “the process by which a strand of nucleic acid joins with a complementary strand through base pairing” as well as the process of amplification as carried out in polymerase chain reaction (PCR) technologies.

The present invention also encompasses the use of nucleotide sequences that are capable of hybridising to the sequences that are complementary to the sequences presented herein, or any derivative, fragment or derivative thereof.

The term “variant” also encompasses sequences that are complementary to sequences that are capable of hybridising to the nucleotide sequences presented herein.

Preferably, the term “variant” encompasses sequences that are complementary to sequences that are capable of hybridising under stringent conditions (e.g. 50° C. and 0.2×SSC {1×SSC=0.15 M NaCl, 0.015 M Na₃citrate pH 7.0}) to the nucleotide sequences presented herein.

More preferably, the term “variant” encompasses sequences that are complementary to sequences that are capable of hybridising under high stringent conditions (e.g. 65° C. and 0.1×SSC {1×SSC=0.15 M NaCl, 0.015 M Na₃citrate pH 7.0}) to the nucleotide sequences presented herein.

The present invention also relates to nucleotide sequences that can hybridise to the nucleotide sequences of the present invention (including complementary sequences of those presented herein).

The present invention also relates to nucleotide sequences that are complementary to sequences that can hybridise to the nucleotide sequences of the present invention (including complementary sequences of those presented herein).

Also included within the scope of the present invention are polynucleotide sequences that are capable of hybridising to the nucleotide sequences presented herein under conditions of intermediate to maximal stringency.

In a preferred aspect, the present invention covers nucleotide sequences that can hybridise to the nucleotide sequence of the present invention, or the complement thereof, under stringent conditions (e.g. 50° C. and 0.2×SSC).

In a more preferred aspect, the present invention covers nucleotide sequences that can hybridise to the nucleotide sequence of the present invention, or the complement thereof, under high stringent conditions (e.g. 65° C. and 0.1×SSC).

### Expression Vector

The terms “plasmid”, “vector system” or “expression vector” means a construct capable of in vivo or in vitro expression. In the context of the present invention, these constructs may be used to introduce genes encoding antibodies into host cells. Suitably, the genes whose expression is introduced may be referred to as “expressible transgenes”.

Preferably, the expression vector is incorporated into the genome of a suitable host organism. The term “incorporated” preferably covers stable incorporation into the genome.

The nucleotide sequences described herein including the nucleotide sequence of the present invention may be present in a vector in which the nucleotide sequence is operably linked to regulatory sequences capable of providing for the expression of the nucleotide sequence by a suitable host organism.

The vectors for use in the present invention may be transformed into a suitable host cell as described below to provide for expression of a polypeptide of the present invention.

The choice of vector eg. a plasmid, cosmid, or phage vector will often depend on the host cell into which it is to be introduced.

The vectors for use in the present invention may contain one or more selectable marker genes—such as a gene, which confers antibiotic resistance eg. ampicillin, kanamycin, chloramphenicol or tetracyclin resistance. Alternatively, the selection may be accomplished by co-transformation (as described in WO91/17243).

Vectors may be used in vitro, for example for the production of RNA or used to transfect, transform, transduce or infect a host cell.

Thus, in a further embodiment, the invention provides a method of making nucleotide sequences of the present invention by introducing a nucleotide sequence of the present invention into a replicable vector, introducing the vector into a compatible host cell, and growing the host cell under conditions which bring about replication of the vector.

The vector may further comprise a nucleotide sequence enabling the vector to replicate in the host cell in question. Examples of such sequences are the origins of replication of plasmids pUC19, pACYC177, pUB110, pE194, pAMB1, pIJ702 and pET11.

### Regulatory Sequences

In some applications, the nucleotide sequence for use in the present invention is operably linked to a regulatory sequence which is capable of providing for the expression of the nucleotide sequence, such as by the chosen host cell. By way of example, the present invention covers a vector comprising the nucleotide sequence of the present invention operably linked to such a regulatory sequence, i.e. the vector is an expression vector.

The term “operably linked” refers to a juxtaposition wherein the components described are in a relationship permitting them to function in their intended manner. A regulatory sequence “operably linked” to a coding sequence is ligated in such a way that expression of the coding sequence is achieved under condition compatible with the control sequences.

The term “regulatory sequences” includes promoters and enhancers and other expression regulation signals.

The term “promoter” is used in the normal sense of the art, e.g. an RNA polymerase binding site.

Enhanced expression of the nucleotide sequence encoding the antibody of the present invention may also be achieved by the selection of heterologous regulatory regions, e.g. promoter, secretion leader and terminator regions.

Preferably, the nucleotide sequence according to the present invention is operably linked to at least a promoter.

Examples of suitable promoters for directing the transcription of the nucleotide sequence in a bacterial, fungal or yeast host are well known in the art.

### Constructs

The term “construct”—which is synonymous with terms such as “conjugate”, “cassette” and “hybrid”—includes a nucleotide sequence for use according to the present invention directly or indirectly attached to a promoter.

An example of an indirect attachment is the provision of a suitable spacer group such as an intron sequence, such as the Sh1-intron or the ADH intron, intermediate the promoter and the nucleotide sequence of the present invention. The same is true for the term “fused” in relation to the present invention which includes direct or indirect attachment. In some cases, the terms do not cover the natural combination of the nucleotide sequence coding for the protein ordinarily associated with the wild type gene promoter and when they are both in their natural environment.

The construct may even contain or express a marker, which allows for the selection of the genetic construct.

For some applications, preferably the construct of the present invention comprises at least the nucleotide sequence of the present invention operably linked to a promoter.

### Host Cells

The term “host cell”—in relation to the present invention includes any cell that comprises either the nucleotide sequence or an expression vector as described above and which is used in the recombinant production of an antibody having the specific properties as defined herein or in the methods of the present invention.

Thus, a further embodiment of the present invention provides host cells transformed or transfected with a nucleotide sequence that expresses the antibodies described in the present invention. The cells will be chosen to be compatible with the said vector and may for example be prokaryotic (for example bacterial), fungal, yeast or plant cells. Preferably, the host cells are not human cells.

Examples of suitable bacterial host organisms are gram positive or gram negative bacterial species.

Depending on the nature of the nucleotide sequence encoding the antibody of the present invention, and/or the desirability for further processing of the expressed protein, eukaryotic hosts such as yeasts or other fungi may be preferred. In general, yeast cells are preferred over fungal cells because they are easier to manipulate. However, some proteins are either poorly secreted from the yeast cell, or in some cases are not processed properly (e.g. hyperglycosylation in yeast). In these instances, a different fungal host organism should be selected.

The use of suitable host cells—such as yeast, fungal and plant host cells—may provide for post-translational modifications (e.g. myristoylation, glycosylation, truncation, lapidation and tyrosine, serine or threonine phosphorylation) as may be needed to confer optimal biological activity on recombinant expression products of the present invention.

The genotype of the host cell may be modified to improve expression.

### Culturing and Production

Host cells transformed with the nucleotide sequence of the present invention may be cultured under conditions conducive to the production of the encoded antibody and which facilitate recovery of the antibody from the cells and/or culture medium.

The medium used to cultivate the cells may be any conventional medium suitable for growing the host cell in questions and obtaining expression of the antibody.

The protein produced by a recombinant cell may be displayed on the surface of the cell.

The antibody may be secreted from the host cells and may conveniently be recovered from the culture medium using well-known procedures.

### Detection

A variety of protocols for detecting and measuring antibodies and binding to their corresponding antigens are known in the art. Examples include antibody-linked immunosorbent assay (ELISA), radioimmunoassay (RIA) and fluorescent activated cell sorting (FACS).

A wide variety of labels and conjugation techniques are known by those skilled in the art and can be used in various nucleic and amino acid assays.

A number of companies such as Pharmacia Biotech (Piscataway, N.J.), Promega (Madison, Wis.), and US Biochemical Corp (Cleveland, Ohio) supply commercial kits and protocols for these procedures.

Suitable reporter molecules or labels include those radionuclides, enzymes, fluorescent, chemiluminescent, or chromogenic agents as well as substrates, cofactors, inhibitors, magnetic particles and the like. Patents teaching the use of such labels include U.S. Pat. No. 3,817,837; U.S. Pat. No. 3,850,752; U.S. Pat. No. 3,939,350; U.S. Pat. No. 3,996,345; U.S. Pat. No. 4,277,437; U.S. Pat. No. 4,275,149 and U.S. Pat. No. 4,366,241.

### Uses of Antibodies According to the Present Invention

Antibody molecules according to the present invention, preferably Fab molecules may be employed in in vivo therapeutic and prophylactic applications, in vitro and in vivo diagnostic applications, in vitro assay and reagent applications, in functional genomics applications and the like.

Therapeutic and prophylactic uses of antibodies and compositions according to the invention involve the administration of the above to a recipient mammal, such as a human. Preferably they involve the administration to the intracellular environment of a mammal.

Substantially pure antibodies of at least 90 to 95% homogeneity are preferred for administration to a mammal, and 98 to 99% or more homogeneity is most preferred for pharmaceutical uses, especially when the mammal is a human. Once purified, partially or to homogeneity as desired, the immunoglobulin molecules may be used diagnostically or therapeutically (including extracorporeally) or in developing and performing assay procedures using methods known to those skilled in the art.

In the instant application, the term “prevention” involves administration of the protective composition prior to the induction of the disease. “Suppression” refers to administration of the composition after an inductive event, but prior to the clinical appearance of the disease. “Treatment” involves administration of the protective composition after disease symptoms become manifest.

The selected antibodies molecules of the present invention can bind Her2/neu positive cells causing receptor function downmodulation in vivo and thus will typically find use in preventing, suppressing or treating cancer. In particular, the antibodies of the invention can reduce proliferation of target cells expressing Her2/neu.

“Target cell” shall mean any undesirable cell in a subject (e.g., a human or animal) that can be targeted by a composition (e.g., a human monoclonal antibody, a bispecific or a multispecific molecule) of the invention. In preferred embodiments, the target cell is a cell expressing, preferably overexpressing, HER2/neu. Cells expressing HER2/neu typically include tumor cells, including adenocarcinoma cells, e.g. salivary gland, stomach and kidney, a mammary gland carcinoma cells, lung carcinoma cells, squamous cell carcinoma cells, and ovarian cancer cells.

Animal model systems which can be used to screen the effectiveness of the selected antibodies of the present invention in protecting against or treating disease are available. Suitable models of cancer will be known to those skilled in the art.

Generally, the selected antibodies of the present invention will be utilised in purified form together with pharmacologically appropriate carriers. Typically, these carriers include aqueous or alcoholic/aqueous solutions, emulsions or suspensions, any including saline and/or buffered media. Parenteral vehicles include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition).

The selected antibodies of the present invention may be used as separately administered compositions or in conjunction with other agents. These can include various immunotherapeutic drugs, such as cylcosporine, methotrexate, adriamycin or cisplatinum, and immunotoxins. Pharmaceutical compositions can include “cocktails” of various cytotoxic or other agents in conjunction with antibodies of the present invention or even combinations of the antibodies, according to the present invention.

The route of administration of pharmaceutical compositions according to the invention may be any of those commonly known to those of ordinary skill in the art. For therapy, including without limitation immunotherapy, the selected antibodies of the invention can be administered to any patient in accordance with standard techniques. The administration can be by any appropriate mode, including parenterally, intravenously, intramuscularly, intraperitoneally, transdermally, via the pulmonary route, or also, appropriately, by direct infusion with a catheter. The dosage and frequency of administration will depend on the age, sex and condition of the patient, concurrent administration of other drugs, counterindications and other parameters to be taken into account by the clinician.

The selected antibodies of the present invention can be lyophilised for storage and reconstituted in a suitable carrier prior to use. Known lyophilisation and reconstitution techniques can be employed. It will be appreciated by those skilled in the art that lyophilisation and reconstitution can lead to varying degrees of functional activity loss and that use levels may have to be adjusted upward to compensate.

The compositions containing the present selected antibodies of the present invention or a cocktail thereof can be administered for prophylactic and/or therapeutic treatments. In certain therapeutic applications, an adequate amount to accomplish at least partial inhibition, suppression, modulation, killing, or some other measurable parameter, of a population of selected cells is defined as a “therapeutically-effective dose”. Amounts needed to achieve this dosage will depend upon the severity of the disease and the general state of the patient's own immune system, but generally range from 0.005 to 5.0 mg of selected immunoglobulin per kilogram of body weight, with doses of 0.05 to 2.0 mg/kg/dose being more commonly used. For prophylactic applications, compositions containing the present selected immunoglobulin molecules or cocktails thereof may also be administered in similar or slightly lower dosages.

A composition containing one or more selected antibody molecules according to the present invention may be utilised in prophylactic and therapeutic settings to aid in the alteration, inactivation, killing or removal of a select target cell population in a mammal. In addition, the selected repertoires of polypeptides described herein may be used extracorporeally or in vitro selectively to kill, deplete or otherwise effectively remove a target cell population from a heterogeneous collection of cells. Blood from a mammal may be combined extracorporeally with the selected antibodies, cell-surface receptors or binding proteins thereof whereby the undesired cells are killed or otherwise removed from the blood for return to the mammal in accordance with standard techniques.

The present invention is further described by way of example, and with reference to the following figures wherein:

### FIGURES

FIG. 1: Soluble Expression of Her2/neu ECD in Yeast Pichia pastoris.

A) Schematic representation of the constructed plasmid for soluble Her2/neu-ECD expression: The cDNA coding for the Her2/neu-ECD was subcloned between Cla I and Xba I restriction sites of the pPICZαC expression vector. The ECD was led by the signal peptide from S. cerevisiae α-factor, which is cleaved by host enzymes while at the C-terminal, it was fused to the c-myc epitope and six His-residue-tag.

B) Purification of the ECD using Ni⁺²-NTA affinity chromatography: Samples corresponding to eluates with increasing imidazole concentrations (40, 60 and 80 mM) were analyzed in 8% SDS PAGE. Proteins were silver stained and molecular weight markers are indicated.

(C, D) Deglycosylation of ECD by peptide N-glycosidase F (PNGase F): Purified ECD incubated for 1 h at 37° C. with or without PNGase F was analyzed in 8% SDS-PAGE followed by western blot, using the anti-myc mAb9E10 (C) or silver stained (D).

E) ELISA test for ECD recognition. Serial dilutions of mAb TAB250 were assessed for binding to 10 μg/ml of purified recombinant ECD.

FIG. 2. Isolation and Characterization of Anti-Her2/neu Fab63 from a Breast Cancer Combinatorial Library.

A) Panning of a phage display library: After the fourth round of panning there is a dramatic increase in phage titers, indicating phage enrichment.

B) Specific binding of Fab63 HER2/neu-ECD in ELISA: Fab63 showed no significant cross-reaction with bovine serum albumin (BSA), human serum albumin (HSA) or the alpha1-ECD of human AChR (α1-AChR). All antigens coated ELISA plates at a concentration of 10 μg/ml. Anti-AChR: a human Fab against the α1-ECD of human AChR

FIGS. 3. A+B Sequence Homology of Human Fab63 with Germline Sequences:

cDNA and deduced amino acid sequences of Fab63 VH (A) and VL (B) regions. Sequences are aligned to the most homologous human germline gene. Dashes incidate nucleotide sequence identity. CDRs are indicated.

FIGS. 3. C+D cDNA Sequences of Fab63 VH (C) and VL (D)

FIG. 4. Fab63 Specificity to Native Her2/neu Receptor:

A) Sandwich ELISA with soluble Her2/neu receptor extracted from SKBR3 cells. The Her2/neu was captured in ELISA plates by anti-Her2 mouse mAb (TAB250) coated wells (0.5 μg/ml). Herceptin antibody was used as a positive control and an anti-huAChR Fab as a negative control. Fabs and Herceptin were detected by goat anti-F(ab′)₂-AP conjugated mAb. The signal for Fab63 was significantly higher upon the presence of Her2/neu receptor

B) FACS analysis of Fab63 binding to positive and negative Her2/neu expressing cells. At the upper panel, Fab63 binds well to Her2/neu expressing SKBR3 cells compared to the positive antibody Herceptin (black histograms) and to cell background (white histograms). Fab63 and Herceptin showed no significant binding to negative Her2/neu HeLa cell line (lower panel).

C) Co-immunoprecipitation of Fab63 with Her2/neu receptor. Western blot analysis of immunoprecipitates, indicates the presence of the Her2/neu protein (185 kDa) and that of Herceptin and Fab63 light chain (˜25 kDa).

D) Competition assay between Fab63 and Herceptin (captured antibody) for Her2/neu receptor binding by sandwich ELISA. Preincubation of Fab63 (550 nM) with Her2/neu receptor inhibits receptor binding to Herceptin up to 88%, while Herceptin acts as an autoinhibitor at much lower concentrations (66 nM). There is no change with anti-Her2/neu IgM and anti-α1 human AChR Fab. Percentage of inhibition was deducted from Her2neu receptor binding to Herceptin without the presence of inhibitors. Inhibitor concentration is showed in a logarithmic scale (χ axis).

FIG. 5. Internalization of Fab63 in SKBR3 Cells.

Immunofluorescence detection of Fab63-treated permeabilized cells resulted in an intense staining of cell cytoplasm after a 2 hour incubation at 37° C. Herceptin signal, under the same conditions, was localized at the membrane while cells treated with just medium showed insignificant fluorescence labeling. Primary Fab/Ab were detected with a-human Kappa light chain/FITC and the results were analyzed by confocal microscopy.

FIG. 6. Time Course Internalization of Fab63 in SKBR3 Cells.

Intracellular staining of Fab63 is observed within 30 min of incubation and reaches highest intensity at 2 hour incubation of cells at 37° C. after acetone treatment. Fab63 is also detectable after 5 hour incubation. Herceptin treatment and immunofluorescence, under the same conditions, showed only membrane staining of cells. Detection of Fab63 was localized at cell membrane, when experiment was performed at 4° C. and when the permeabilization step was omitted. Under no permeabilization treatment of cells, and Fab63 signal was detected at the membrane

FIG. 7. Fab63 Inhibits Proliferation of Her2/neu-Positive MDA-MB-453 and SKBR3 Cells.

A) Dose response curves for Her2/neu expressing positive cell lines (MDA-MB-453 and SKBR3) or Her2/neu negative (MDA-MB-435) cells. The cell survival was expressed as percentage of live cells compared to untreated cells. Fab63 showed growth inhibition only on high Her2.neu expressing cells.

B) Comparison of Fab63 with Herceptin on the growth of Her2/neu-positive (MDA-MB-453, SKBR3) and negative (MDA-MB-435) cell lines.

FIG. 8. Effects of Fab63 on Cancer Cell Lines in the Presence and Absence of HRG-β1 Ligand.

MDA-MB-453 (A) and MCF7 (B) cancer cells were treated with 250 nM of Fab63 or 66 nM Herceptin. Fab63 and Herceptin both inhibit cell growth in the high Her2/neu expressing cells MDA-MB-453 and not in the low expressing MCF7, in the absence of the growth factor, while in the presence of HRG-β1 only Fab63 can produce a significant growth inhibition effect in both cell lines.

### SEQUENCE LISTING

SEQ ID NO: 1—amino acid sequence of CDR1 of VH Fab63
SEQ ID NO: 2—amino acid sequence of CDR2 of VH Fab63
SEQ ID NO: 3—amino acid sequence of CDR3 of VH Fab63
SEQ ID NO: 4—amino acid sequence of CDR1 of VL Fab63
SEQ ID NO: 5—amino acid sequence of CDR2 of VL Fab63
SEQ ID NO: 6—amino acid sequence of CDR3 of VL Fab63
SEQ ID NO: 7—amino acid sequence of VH Fab63
SEQ ID NO: 8—amino acid sequence of VL Fab63
SEQ ID NO: 9—nucleic acid sequence of CDR1 of VH Fab63
SEQ ID NO: 10—nucleic acid sequence of CDR2 of VH Fab63
SEQ ID NO: 11—nucleic acid sequence of CDR3 of VH Fab63
SEQ ID NO: 12—nucleic acid sequence of CDR1 of VL Fab63
SEQ ID NO: 13—nucleic acid sequence of CDR2 of VL Fab63
SEQ ID NO: 14—nucleic acid sequence of CDR3 of VL Fab63
SEQ ID NO: 15—nucleic acid sequence of VH Fab63
SEQ ID NO: 16—nucleic acid sequence of VL Fab63

### EXAMPLES

### General Methods

The methods described here may employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; Using Antibodies: A Laboratory Manual: Portable Protocol NO. I by Edward Harlow, David Lane, Ed Harlow (1999, Cold Spring Harbor Laboratory Press, ISBN 0-87969-544-7); Antibodies: A Laboratory Manual by Ed Harlow (Editor), David Lane (Editor) (1988, Cold Spring Harbor Laboratory Press, ISBN 0-87969-314-2), 1855. Handbook of Drug Screening, edited by Ramakrishna Seethala, Prabhavathi B. Fernandes (2001, New York, N.Y., Marcel Dekker, ISBN 0-8247-0562-9); and Lab Ref: A Handbook of Recipes, Reagents, and Other Reference Tools for Use at the Bench, Edited Jane Roskams and Linda Rodgers, 2002, Cold Spring Harbor Laboratory, ISBN 0-87969-630-3, “Monoclonal antibodies from combinatorial Libraries” 1994 Cold Spring Harbor Laboratory, C. F. Barbas and D. R. Burton Each of these general texts is herein incorporated by reference.

### Materials and Methods

Plasmid Construction and Soluble Expression of Her2/neu Extracellular Domain in Yeast Pichia pastoris.

The N-terminal extracellular domain (ECD) of human Her2/neu oncogene (1881 bp fragment, aminoacid residues 1-627) was enzymatically amplified by PCR using a full-length cDNA clone (kindly provided by Dr Mien-Chie Hung). The upstream primer (5′-CTCTTGCCCCCCGGAATCGATAGCACCCAAGTGTGC-3′) and the downstream primer (5′-GAGATGATGGACGTCTCTAGACTGGCTCTCTGCTCG-3′) were constructed to contain the ClaI and XbaI restriction sites, respectively (underlined). Using the appropriate restriction endonucleases, the purified cDNA fragment was subcloned into the expression vector pPICZαC (Invitrogen). The recombinant fragment was preceded by a signal peptide α-factor under the transcriptional control of the AOX promoter, which is induced by methanol, whereas the C-terminal end, it was fused to a sequence encoding the c-Myc epitope and polyhistidine (His6) tag. The linearized construct was electroporated into the P. pastoris and the transformed cells were plated on YPDS (1% yeast extract, 2% peptone, 2% dextrose, and 1 M sorbitol) plus zeocin (100 μg/ml) and incubated at 30° C. for 3 days. Transformants were screened by PCR using 5′α factor and 3′AOX1 primers (Invitrogen). Individual clones were cultured in BMGY, BMGH or MGYH medium. After 16-20 h incubation, the cells were collected by centrifugation (3000×g for 5 min at room temperature) and resuspended in BMMY, BMMH or MMH medium respectively (consisting of BMGY, BMGH or MGYH medium with 0.5% methanol instead of 1% glycerol) to start induction. Expression was induced with addition of 0.5% v/v methanol, for the first and second day, while 1% v/v was added for the third and forth day. At the end of the induction the culture was harvested by centrifugation (8000×g for 20 min at 4° C.) to remove cells and debris. The culture supernatants were tested for expression of Her2/neu-ECD by dot blot analysis using the anti-Myc 9E.10 mAb (ATCC). The clone with the highest protein yield was used for large-scale protein expression.

Purification of ECD from P. pastoris Culture Supernatant

The culture supernatant was passed through a 0.22-μm membrane filter, followed by the addition of PMSF (1 mM final concentration) and NaN₃ (0.05% final concentration). The filtrate was subsequently concentrated to 20 fold, using the Minitan Ultrafiltration System (Millipore, Bedford, Mass.) equipped with 30 kDa cut-off membrane and dialyzed extensively against phosphate buffer (5.8 mM KH₂PO₄, 40 mM Na₂PO₄, pH 8). The concentrated protein was then purified using Ni²⁺-NTA affinity chromatography (QIAgen, Germany), under native conditions, according to the manufacturer's protocol. To prevent non-specific interactions, binding and washing procedures were performed in a high salt concentration (2 M NaCl) and low imidazole concentration (10 mM) phosphate buffer, pH 8. Ni²⁺-NTA agarose, equilibrated in the same buffer, was added to the dialyzed supernatant and incubated under gentle rotation for 16 h at 4° C. Elution of the recombinant protein was performed using increasing imidazole concentrations (40, 60, 80 and 100 mM). Protein concentration was determined in eluates with the Coomasie plus protein assay reagent kit (Pierce, Rockford, Ill.) following the manufacturer's instructions, using bovine serum albumin as a standard. The purity of the Her2/neu-ECD was estimated with SDS-PAGE.

In Vitro Deglycosylation of Recombinant Her2/neu-ECD.

Post-translational modification of recombinant ECD from P. pastoris was analyzed in a deglycosylation reaction. Approximately 7 μg of ECD were denatured at 100° C. for 10 min in a buffer containing 0.5% SDS and 1% β-mercaptoethanol. 50 mM sodium phosphate and NP to a final concentration of 1% were added, and the protein was incubated with 1000 U N-glycosidase F (PNGase F, New England Biolabs) for 1 h at 37° C., in a total reaction volume of 160 μl. PNGase F has an apparent molecular weight of 36 kDa.

### Cell Lines and Culture.

A panel of cell lines was used as breast tumour models revised previously (Lacroix and Leclercq, 2004). These include SKBR3 and MDA-MB-453 that overexpress Her2/neu protein and the low-expressing Her2/neu, MDA-MB-435 and MCF7. These cells as well as HeLa cells were purchased from the American Type Culture Collection and maintained in DMEM or RPMI-1640 supplemented with 10% FBS (Gibco BRL) at 37° C. under a 5% CO₂-95% air atmosphere.

### Construction and Biopanning of the Combinatorial Human Fab-Phage Library

Lymphocytes from a patient with Her2/neu positive breast tumor were isolated from invaded lymph nodes using Lymphoprep Ficoll (Sigma, Saint Louis, Mo.) and then the B cell population was subtracted using an anti-CD19 column (Miltenyi Biotech, Cermany). Total RNA was isolated using the guanidium isothyocyanate/acid phenol method (Chomczynski and Sacchi, 1987). Library construction and panning was performed as described previously (Barbas et al., 1991; Burton, 1991; Barbas & Burton, 1994). In short, total RNA from B cells and human Ig-specific degenerated primers for the heavy chain Fd and light chain were used in one step RT-PCR reaction (Access RT-PCR System, Promega). The PCR products were purified and reamplified in order to introduce restriction sites for cloning into pComb3H vector (kindly provided by Dr. C. Barbas and Pr. D. Burton) (Williamson et al., 1993). The amplified Vλ-Cλ, Vκ-Cκ and VH-CH1 cDNA fragments were cloned into pComb3H phagemid between the SacI/XbaI and XhoI/SpeI restriction sites, respectively. The combinatorial library was electroporated into E. coli XL1-Blue cells and packaged with VCS-M13 helper phage as described (Barbas et al., 1991). Phages were precipitated from the supernatant with addition of 4% polyethylene glycol, 0.5 M NaCl and resuspended in 2 ml PBS containing 1% BSA, 0.02% NaN₃.

Library panning was performed in four microtiter plate wells (4×50 μl) coated with the recombinant Her2/neu-ECD (1 μg/well) antigen, expressed in yeast P. pastoris. Plates were blocked and incubated with 50 μl of PEG precipated phages at a concentration of 10¹² cfu/ml, for 2 h at 37° C. Non-specific binders were removed by following a mode of 1×, 3×, 6×, 10× increasing stringency wash steps, whereas bound phages were eluted and re-amplified for the next round of panning. The blocking medium was 3% BSA in PBS, phages were washed with 0.5% Tween20 in PBS, eluted with glycine buffer pH 2.2 and neutralized with 2M Tris base. Phages were stored at 4° C. in 1% BSA in PBS.

### Production and Purification of Soluble Fab Fragment.

Phagemid DNA from the last round of panning was isolated from E. coli, subjected to Spe1/NheI (N.E.Biolabs) digestion for the removal of pIII protein and re-ligated to transform XL1-Blue cells. Bacterial cultures from single colonies were grown at 37° C. to an OD₆₀₀ of ˜1.0, then overnight at 30° C. after addition of 1 mM IPTG (Promega, USA) to induce soluble Fab production. Soluble Fabs were obtained from the periplasm using the freeze-thaw method or sonication to lyse the cells (Barbas et al., 1991).

Soluble Fab was purified by affinity chromatography using a goat anti-human F(ab′)₂ antibody (PIERCE, USA) covalently linked to protein G-coated agarose beads (Sigma-Aldrich, Germany), as described previously (Barbas et al., 1991). Bound Fabs were eluted with 0.2 M glycine-HCl buffer, pH 2.8, followed by 0.2 M glycine-HCl buffer, pH 2.5, both eluates being immediately neutralized with 1 M Tris-HCl, pH 9.0, then dialyzed against PBS, 0.2 mM EDTA.

The eluates were subjected to 12% SDS-PAGE and the proteins were visualized with coomasie or silver stain. Fab was detected in Western blot with the goat anti-human IgG, F(ab′)₂, followed by an anti-goat HRP conjugated (1:1000, DAKO). The concentration of protein was determined by Bradford method (Biorad) and Fab tested in ELISA as described above. Purified Fab was used in all experiments from this point forward.

### DNA Sequencing and Analysis

DNA preparations from Fab expressing clones were sequenced both manually (Sequenase chain-termination DNA method, USB) and automatically in an ALF-Express Sequenator (Sequence facility of the Microchemistry Lab, IMBB, Crete), in both directions using PelB and SEQGz primers for heavy chain and OmpA, SEQκb, and SEQλb primers for light chain (Graus et al., 1997). The germline counterparts of the rearranged VH and VL sequences were analyzed using the National Center for Biotechnology Information IgBLAST server (http://www.ncbi.nlm.nih.gov/igblast/) and sequences were aligned using ClustalW software. Complementarity-determining regions (CDRs) were assigned according to the definition of Kabat (Kabat E. A.).

### Fab Characterization by ELISA and Immunoprecipitation.

Microtiter plates were coated with Her2/neu-ECD or control antigens (10 μg/ml), blocked with 3% BSA in PBS and incubated with crude lysates containing the Fab fragments for 1 h at 37° C. Plates were then washed 10 times with 0.05% Tween20 in PBS and incubated with goat anti-human IgG, F(ab′)₂ alkaline phosphatase conjugated (1:1000, PIERCE), which recognize the human light chain IgG, for 1 h at 37° C. After a final wash as above the substrate solution was added (pNPP, SIGMA) and the plate OD read at 405 nm in an ELISA reader (Biorad).

For sandwich ELISA, purified Fab63 and control antibodies, anti-AChR Fab (Fostieri et al., 2005) and Herceptin, were incubated with solubilized Her2/neu receptor, previously captured in microtiter plates with 0.5 μg/ml mouse anti-Her2 antibody (TAB250, Zymed). Her2/neu receptor was extracted from SKBR3 cells, by homogenizing the cells in lysis buffer (140 mM NaCl, 50 mM NF, 10 mM Tris, 5 mM EDTA, 2 mM EGTA, 5 mM IAA, 0.5 mM PMSF, 5 u/ml aprotinin, 5 μg/ml pepstatin), at 4° C. for 30 min. Subsequently, the centrifuged pellets (14,000 g for 30 min at 4° C.) were washed, resuspended in the same buffer plus 2% Triton X-100 and homogenized with a needle syringe. The samples were incubated at 4° C. overnight, centrifuged as above, and the supernatant containing the solubilized receptor was collected. The final step of Fab or antibody detection was performed as previously described.

For competition ELISA, Her2/neu receptor was initially preincubated with purified Fab63 or competitors-antibodies, overnight at 4° C. and the resulted receptor complexes were captured by 5 μg/ml Fab63 or 1 μg/ml Herceptin IgG in microtiter plates. Receptor was detected by C-18 polyclonal antibody (Santa Cruz) followed by an anti-rabbit HRP (1:1000, DACO) with a final OD reading at 450 nm with the addition of substrate (TMB, Fermentas).

For immunoprecipitation, cells were grown to ˜50% confluency in 6-well plates overnight. Next day, they were washed with PBS and incubated with 500 nM Fab or 66 nMHerceptin for 30 min. Cells were lysed as above and membrane extracts were immunoprecipitated with 10 μl gel slurry of goat anti-human IgG, F(ab′)₂ pre-bound on protein G beads at a final volume of 50 μl for 1-2 hours at room temperature. The beads were washed three times with 0.1% Tween, resuspended in loading buffer, boiled and analysed by Western blot. Her2/neu receptor was detected using C-18 polyclonal antibody, whereas the light chain of Fab and Herceptin were detected by goat anti-human IgG F(ab′)₂ mAb.

### Flow Cytometry Analysis.

Live cells were harvested, washed with PBS and saturated in blocking buffer (1% FBS in PBS). Aliquots of 1×10⁵ cells incubated with Fab63, Herceptin and anti-AChR human Fab diluted in blocking buffer for 1-2 hours at 4° C. Cells were incubated with the goat anti-human IgG, F(ab′)₂ (1:1000 dilution) followed by a FITC-conjugated rabbit anti-goat antibody (1:100, Dako). Cells were washed, resuspended in PBS and analyzed in respect of mean fluorescence intensity (MFI) at a FACScan™ flow cytometer using CellQuest software (Becton-Dickinson).

### Confocal Laser Scanning Microscopy

Semi-confluent SKBR3 cells, grown on poly-1-lysine (Sigma) coated glass lamelles, were washed with PBS and incubated with Fab63 or Herceptin for the indicated time at 4° C. or 37° C. Cells were then fixed with 4% paroformaldehyde in PBS at room temperature for 5 min, while for internalization studies they were permabilized with ice cold acetone for 30 seconds (Poul et al., 2000). Cells were washed and blocked in Mab's buffer (10% FBS, 60 mM Lysine in PBS) for 20 min at 37° C., and fluorescein labelling of Fab63 and Herceptin was performed as in flow cytometry. Finally, lamelles were washed, mounted with Citifluor™ and inverted on slides. Immunofluorescence analysis was performed in a Leica TCS-SP confocal microscope.

### Cell Proliferation Assay.

Single cell suspensions of 10⁵ cells/ml were prepared and left to adhere overnight at 96-well culture plates at 100 μl/well. After a brief wash of cells, we added 100 μl medium supplemented with 10% FBS±Fab63 at a concentration range of 50-550 nM or Herceptin at 66 nM. To test Fab63-Herceptin synergy, cells were cultured in the presence of both agents at maximum concentration ratio. For ligand-activated studies, cells were treated with HRG-β1 (50 ng/ml) 30 min subsequently to the addition of Fab63 or Herceptin in the presence of 10% and 1% FBS for MDA-MB-453 and MCF7 cell lines, respectively. Cells were incubated for 72 hours and the number of living cells was determined by MTT assay (CellTiter 96® AQueous One Solution cell proliferation assay, Promega). Cell survival was expressed as the percent growth of cells cultured without Fab63 or Herceptin.

### Results

Expression and Purification of the Extracellular Domain of Her2 in Yeast P. pastoris

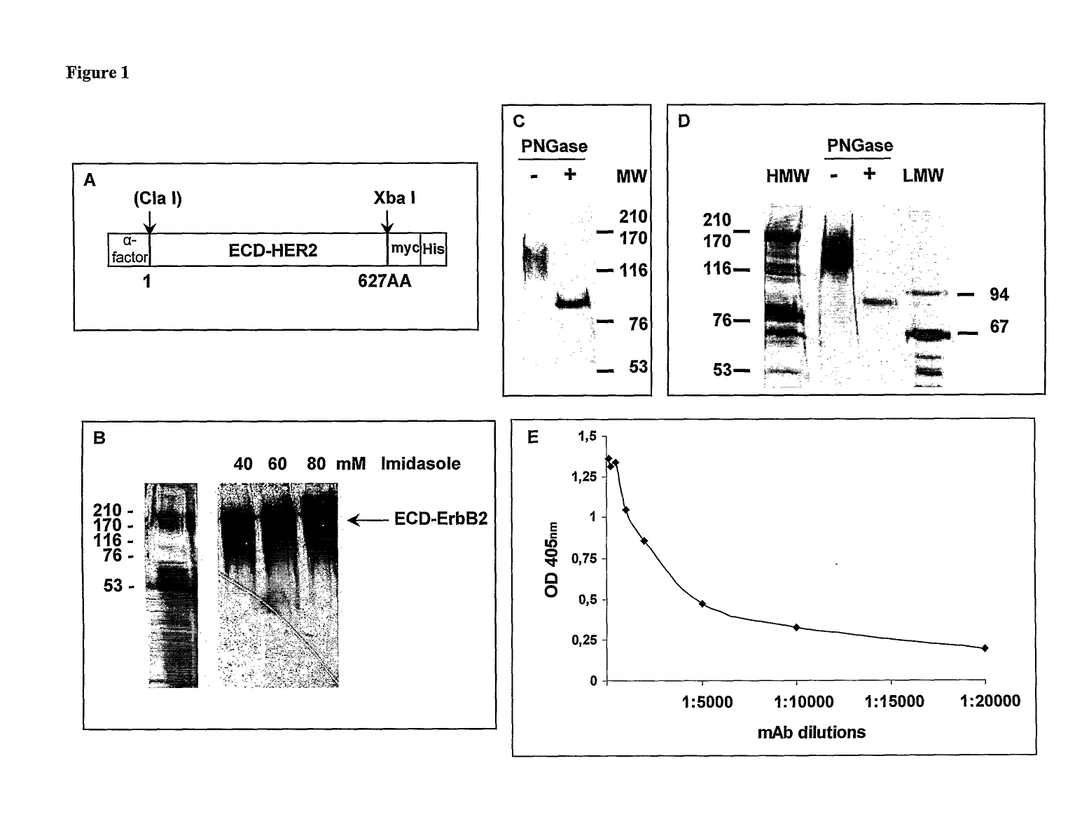

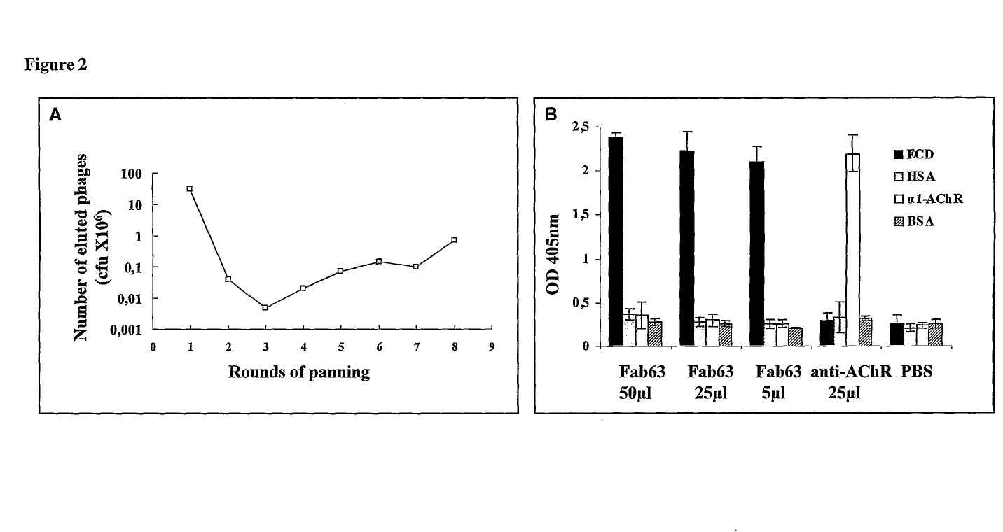

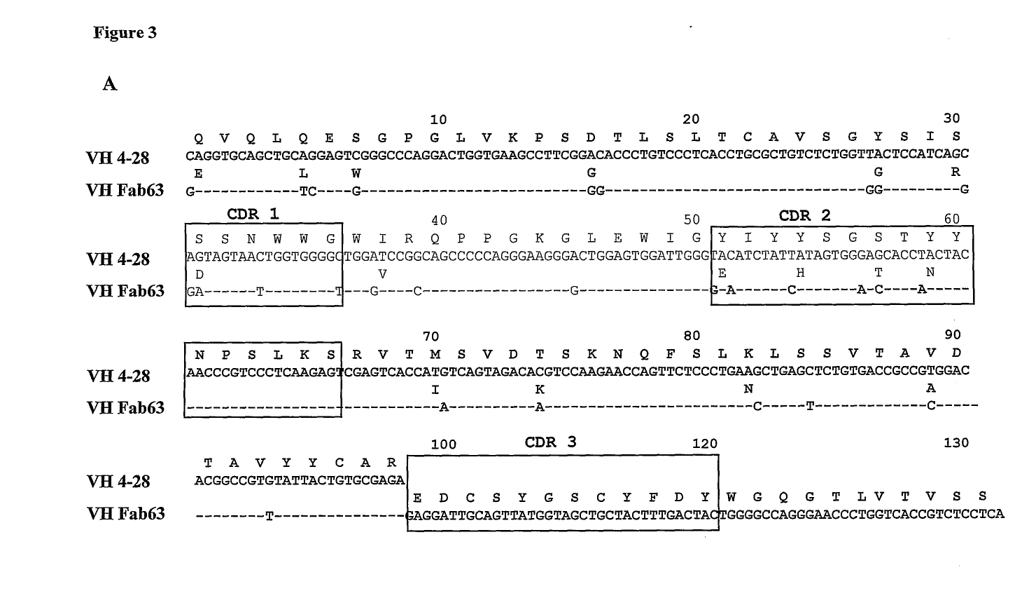

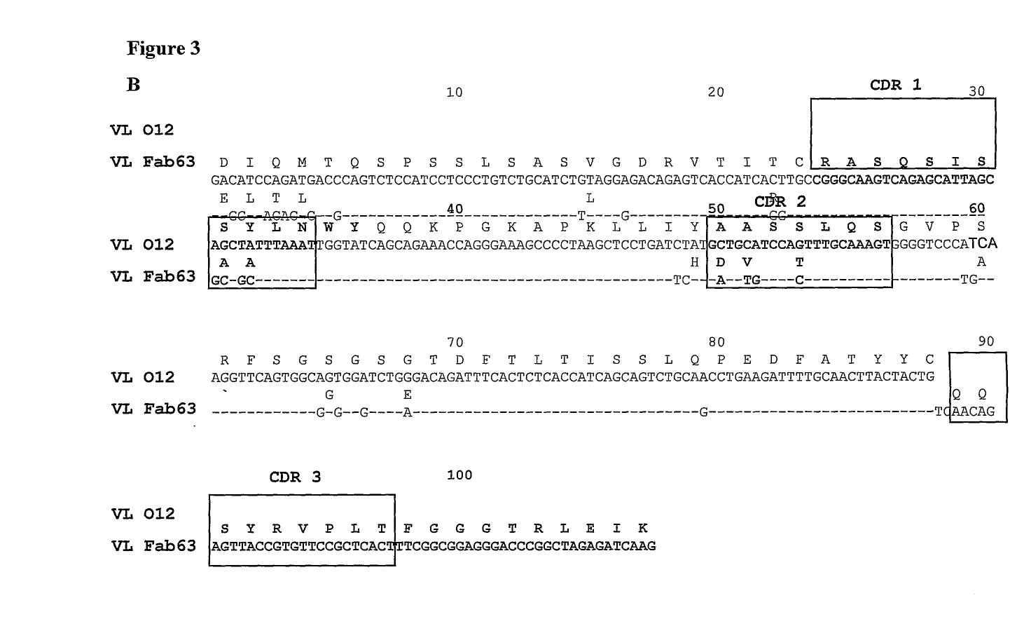

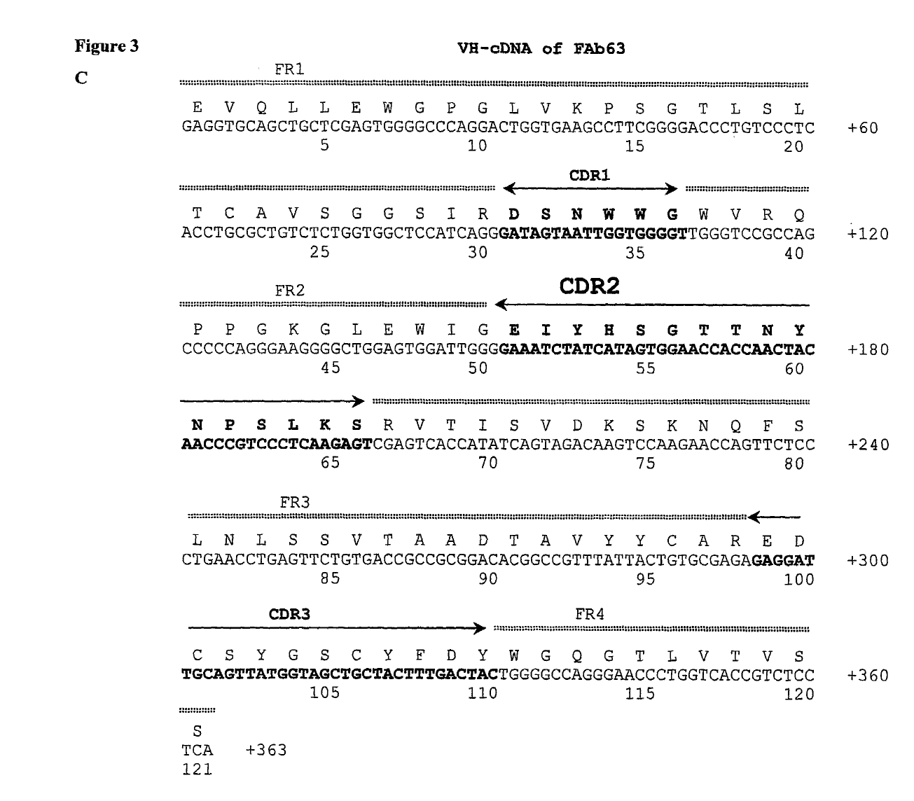

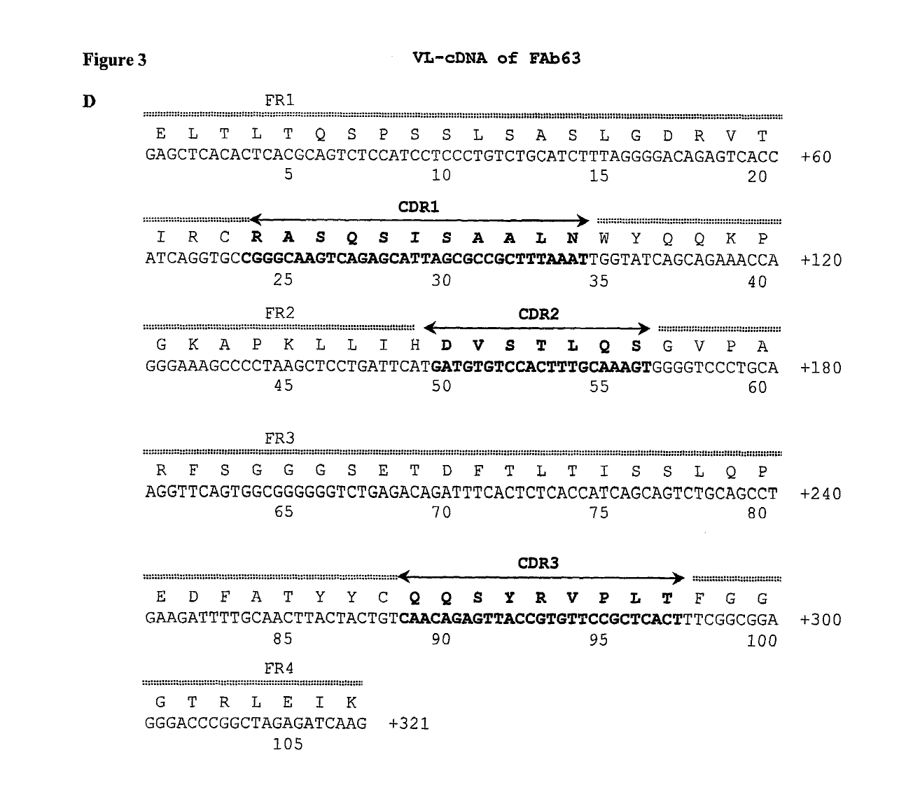

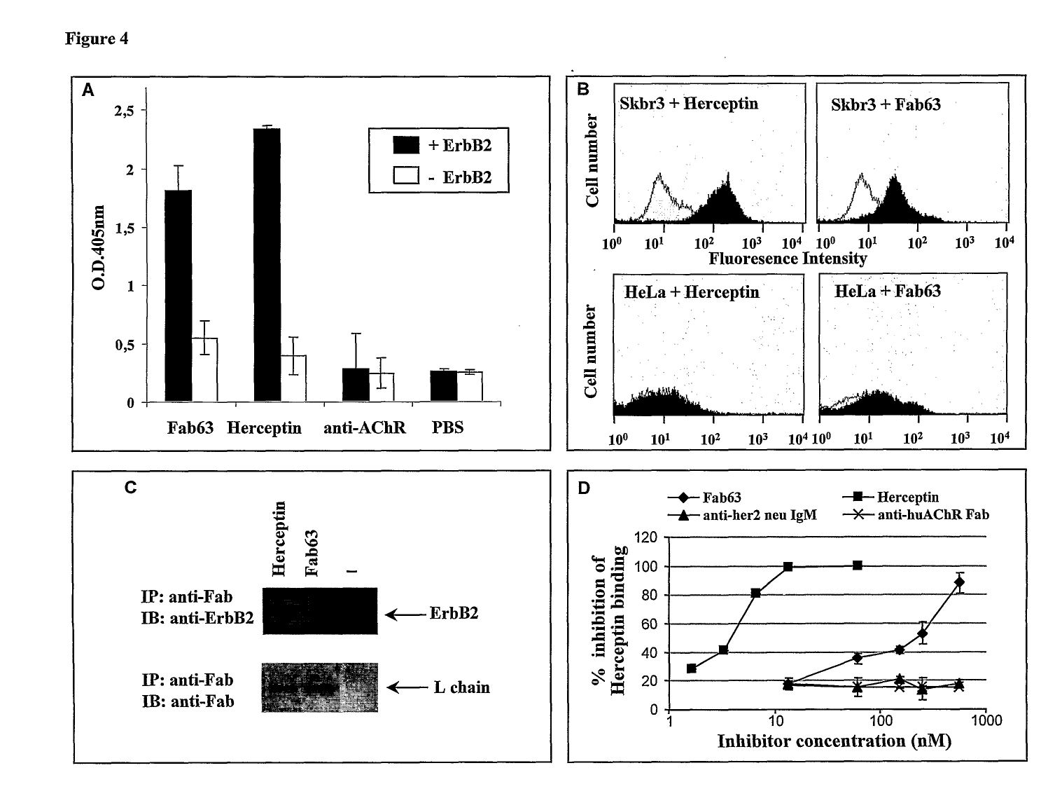

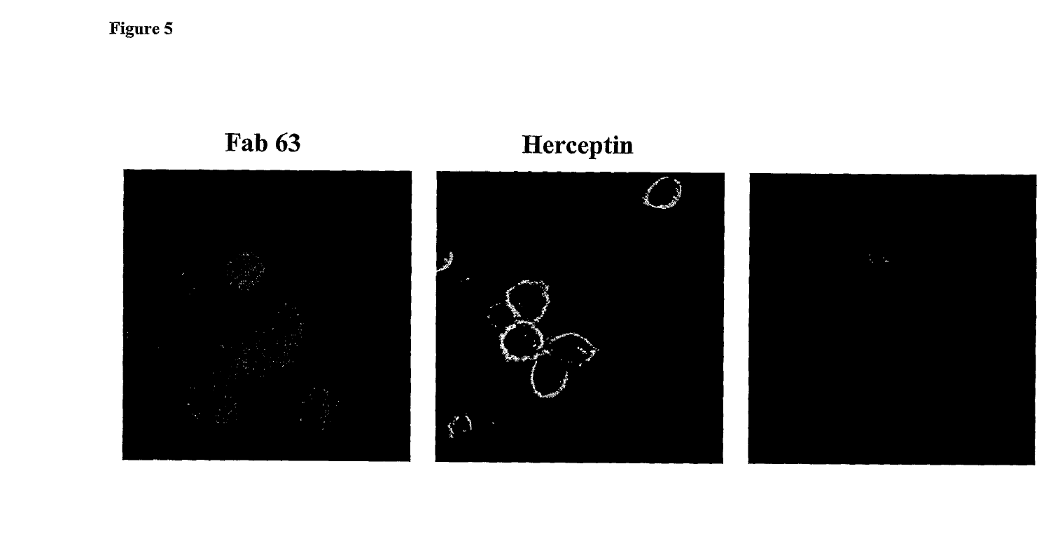

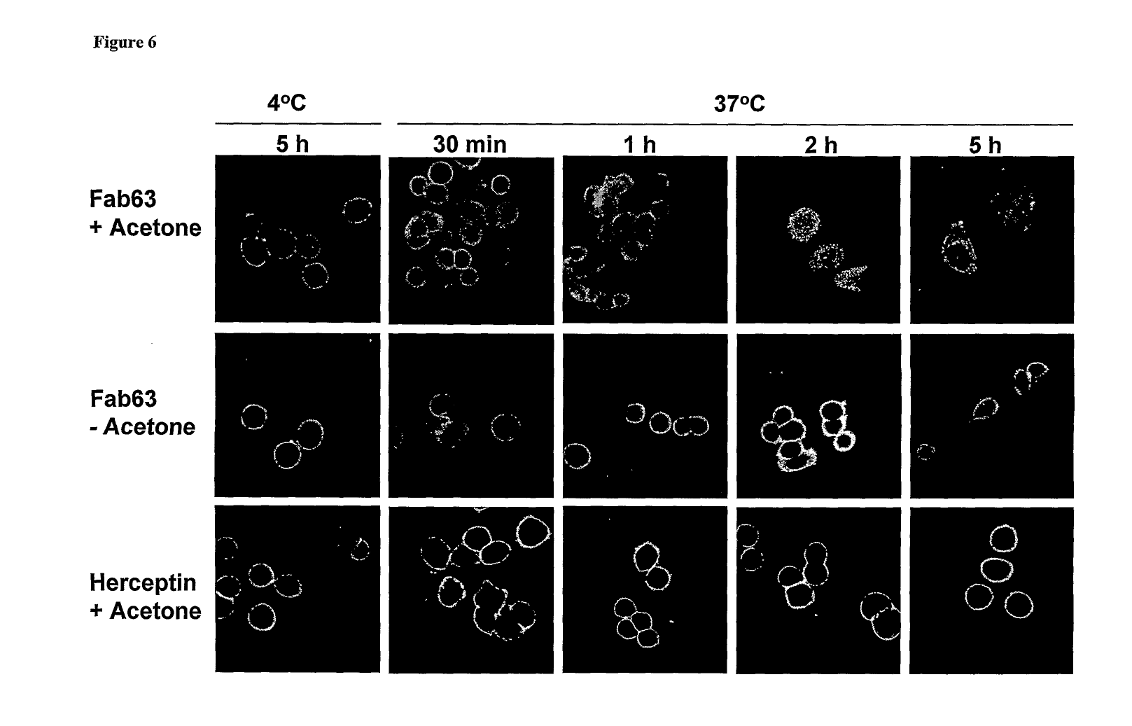

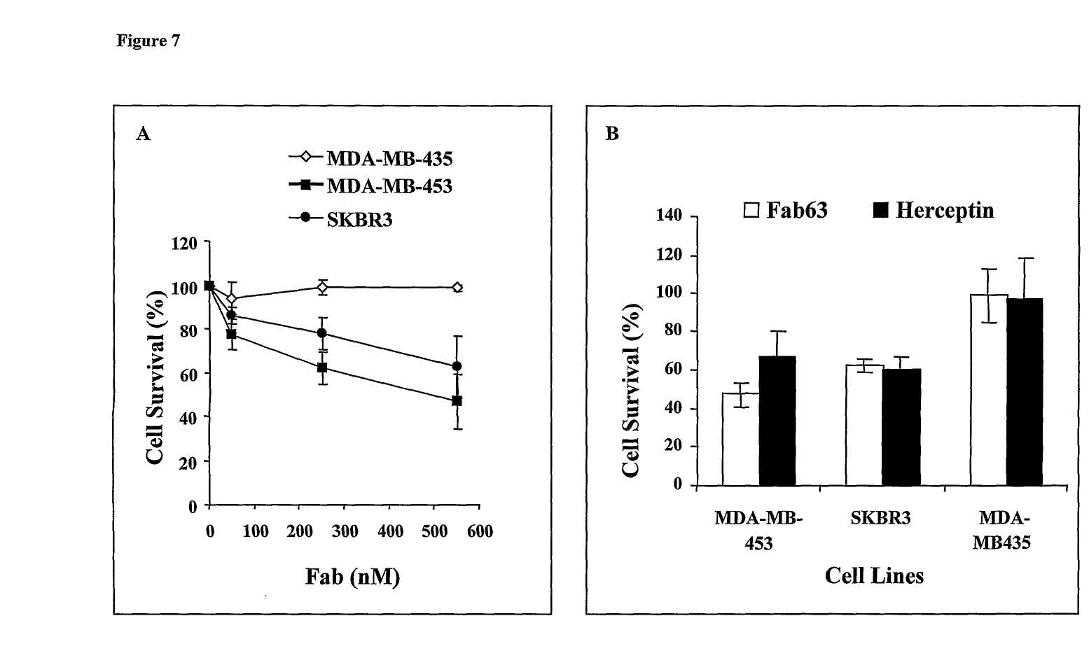

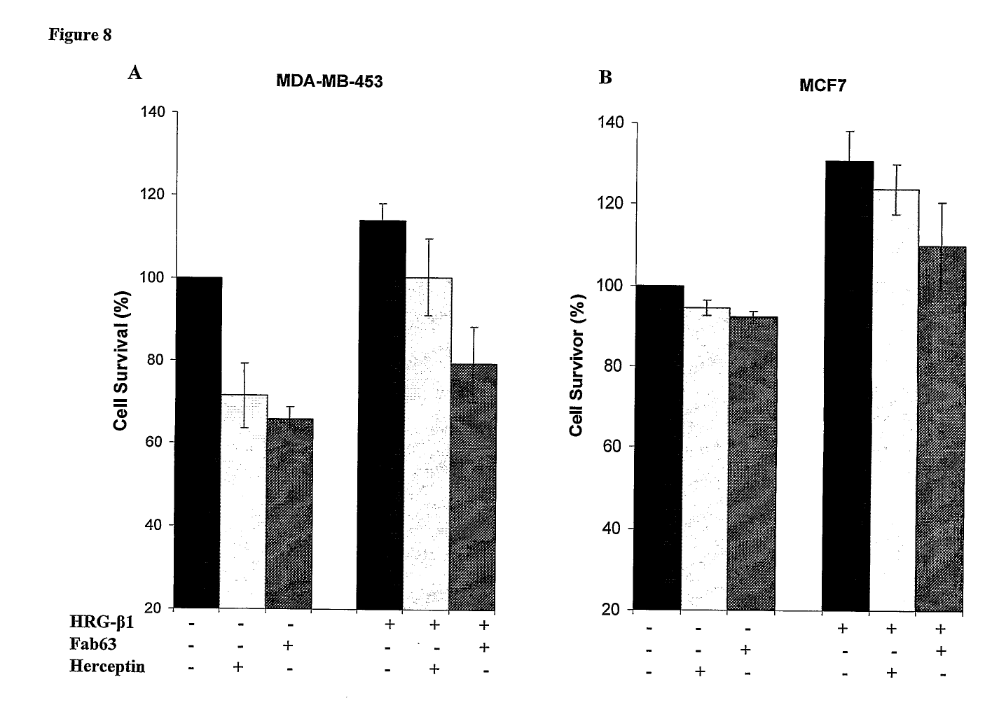

cDNA encoding the extracellular domain (ECD) of Her2 was amplified by PCR and cloned into pPICZαC P. pastoris expression vector for large-scale expression and purification of the protein (FIG. 1A). This construct, pPICZαC-ECD, was linearized and transformed into P. pastoris strains X33 and GS115 and recombinant clones were selected in medium containing zeocin (100 μg/ml). Protein expression was determined by growing individual transformants in three different culture media (BMGY, BMGH and MGYH). Protein production was monitored every 24 h, by adding methanol, for 4 days to determine the length of time required for optimal protein production. Dot blot analysis of culture supernatants revealed that the highest level of expression of Her2-ECD was observed in X33 transformants, after 72 h induction with methanol, in BMGY medium (data not shown). A high expressing clone was selected for further analysis.

The secreted ECD was purified from culture supernatant by affinity chromatography on a Ni²⁺-NTA column. To prevent non-specific interactions, binding and washing procedures were performed in phosphate buffer, pH 8, containing 2 M NaCl and 10 mM imidazole. The protein was eluted using increasing imidazole concentrations (40, 60, 80 and 100 mM). The fractions were analysed by SDS-PAGE and Western blotting. FIG. 1B shows that the majority of Her2-ECD protein is eluted with 40, 60 and 80 mM imidazole. The molecular size of the product was estimated as 120-210 kDa, higher than that predicted from the amino acid sequence (73 kDa). This difference was shown to be due to glycosylation of the molecule since enzymatic deglycosylation using peptide N-glycosidase F resulted in a reduction in the apparent molecular size to about 85 kDa (FIG. 1C). Moreover, the deglycosylated and untreated ECD were detected by Western blotting with both anti-myc (9E10) and anti-human Her2/neu ICR12 mAbs (FIG. 1D). These results show that, like the native protein erbB2, Her2-ECD is glycosylated. The yield of purified ECD was 0.2-0.3 mg/liter. The anti-human Her2/neu mAb ICR12 and the mAbs TAB250 and Herceptin that do not bind to denaturated Her2/neu protein (data not shown), were assessed for binding to the purified recombinant Her2-ECD, in ELISA. All mAbs tested recognized ECD very well, suggesting that the recombinant protein was folded in a native like conformation (FIG. 1E, FIG. 4A).

### Construction of the Combinatorial Human Fab Library—Isolation of the Specific Anti Her2-ECD Fab63.

The phage display Fab library was constructed from B-lymphocytes isolated from invaded lymph nodes of a patient with Her2/neu positive breast cancer. The light chain gene repertoire (κ and λ) was cloned into the pComb3H phagemid and subsequently the Fd heavy chain gene repertoire of the γ1 isotype was inserted into the light chain library. A human combinatorial Fab library with a complexity of 1×10⁷ cfu/μg was created. For selection of specific anti-Her2 Fab antibody fragments the combinatorial library was panned with the recombinant Her2-ECD antigen. The final round of panning yielded a 100 fold amplification in the phage titers compared to the minimum eluates from the third round of panning (FIG. 2A), indicating an enrichment in clones with enhanced affinity towards the specific antigen. Phagemid DNA was isolated from the eight round of panning and modified to produce soluble Fabs. Several clones were tested in ELISA with Her2-ECD, and clone 63 was selected for further characterization.

The specific binding of human Fab 63 was first tested in ELISA assay. FIG. 2B shows that the Fab 63 bound specifically to the Her2-ECD and not to irrelevant antigens such as human serum albumin (HSA), the alpha1-ECD (α1-ECD) of human AChR (Psaridi-Linardaki et al., 2002) (both produced in P. pastoris) or to the bovine serum albumin (BSA).

Soluble Fab63 was produced in large scale by IPTG induction. Fab was visualized and tested for its purity in coomassie and silver stained SDS-PAGE as a double band of approximately 30 kD and was also detected by Western blotting with anti-human Fab antibody (data not shown). Pure Fab63 was quantified by Bradford method and used for all further experiments in PBS. The yield of purified Fab was approximately 1 mg/liter.

The primary structure of the fully human Fab63 was determined by sequencing and compared to germline V genes according to Kabat and Blast databases. The VH-cDNA of Fab63 was closest to VH4 gene family, showing an 83% homology in amino acid analysis (FIG. 3A), while the VL-cDNA belongs to K subgroup and shares an 82% homology with the O2 germline amino acid sequence (FIG. 3B). The cDNA and amino acid sequences of VH and VL of Fab63 are shown in FIGS. 3C and 3D, respectively.

### Fab63 Specificity for Native Her21neu Receptor.

Fab63 was able to bind to soluble Her2/neu whole receptor extracted from the high expressing Her2/neu SKBR3 cell line as described above. The receptor was captured by an anti-Her2/neu mouse antibody specific for an extracellular domain of Her2/neu, in ELISA plates. Fab63 was added at a concentration of 250 nM, together with Herceptin as a positive antibody at 10 nM and an anti-huAChR Fab (anti-AChR) at 250 nM as a negative control. Detection of Fab63 and Herceptin with an anti-human Fab antibody produced a strong signal only in those wells where Her2/neu receptor was captured. Irrelevant Fab showed background binding in any case (FIG. 4A).

We further investigated the ability of Fab63 to bind to intact cells by flow cytometry. Fab63 bound to the positive Her2/neu SKBR3 cell line and produced only background signal in the negative HeLa cell line compared to Herceptin (FIG. 4B) and anti-huAChR Fab (data not shown).

The same results were obtained by using confocal microscopy. Fab63 and Herceptin binding was visualized in SKBR3 cell membrane by staining cells previously grown and fixed on cover slips, at 4° C. (FIG. 6).

The specificity of Fab63 binding to the Her2/neu receptor was further investigated. After treatment of SKBR3 cells with Fab63, it was found that the Her2/neu membrane receptor was co-immunoprecipitated with the antibody fragment (FIG. 4C). In the Western blot, a 185 KDa band was detected by using the Neu C-18 polyclonal Ab, corresponding to Her2/neu protein, whereas the light chain of Fab63 and Herceptin were detected with the anti-human IgG, F(ab′)₂ mAb.

In an attempt to track down the site of Fab63 recognition and test the ability to compete Herceptin, we performed competition assays for binding the soluble Her2/neu receptor. From the commercial anti-Her2/neu non-human antibodies that were used, none was able to influence Fab63 binding to the receptor and only Herceptin competed Fab63 binding at a concentration as low as 2 nM (data not shown). Vice versa, when Fab63 was preincubated with Her2/neu inhibited Herceptin binding to the receptor up to 88% at 550 nM. Herceptin as an autocompetitor inhibited self binding at 100% in a concentration of 66 nM, while an anti-Her2/neu IgM human Ab and an irrelevant anti-huAChR Fab showed no competition (FIG. 4D). These data suggest that the binding sites of Herceptin and Fab63 are in close proximity.

### Fab63 is Internalized in SKBR3 Cells.

The ability of antibodies to internalize into the cell is required for many targeted therapeutics. Initial internalization experiments were set at 2 hour incubation and revealed the strong characteristic of Fab63 to enter into the cell, while Herceptin was detected at the cell membrane (FIG. 5). Subsequent, time-course confocal analysis at 30 minutes, 1, 2 and 5 hours (FIG. 6), showed that Fab63 was rapidly internalized within 30 minutes of incubation with SKBR3. Fab63 exerts its maximum efficiency of internalization after 2 hours at 37° C., exhibiting a strong intracellular signal, while Herceptin stained only the membrane of the cells at the corresponding time intervals. Under no permabilization condition, as well as at 4° C. incubation, Fab63 signal was detected at the surface membranes as it was expected.

### Growth Inhibitory Effects of Human Fab63 on Her2/neu Expressing Cell Lines

To further exploit the functional characteristics of Fab63 to cells, following binding to the receptor and internalization, we studied the effects on cell proliferation using a panel of high and low Her2/neu expressing cancer cell lines. Cells were cultured with increasing concentration of Fab63 and without Fab in order to deduce the normal cell proliferation rate by counting cell number after 72 hours of culture. Fab63 inhibited cell growth up to ˜44% in MDA-MB-453 and ˜37% in SKBR3 high Her2/neu expressing cell lines at the maximum concentration of 500 nM. Growth inhibition was also observed at lower concentrations of 50 and 250 nM showing a dose dependent effect on MDA-MB-453 and SKBR3 while there was no arrest of cell growth in MDA-MB-435, low Her2/neu expressing cell lines, at any concentration (FIG. 7A).

Herceptin is known to arrest cell growth of Her2/neu positive cells in a cytostatic manner and thus was used as positive standard and as a comparative agent for Fab63. Herceptin had a similar behaviour as Fab63, inhibiting cell growth at ˜36% in MDA-MB-453 and at ˜40% in SKBR3 cells at a concentration as low as 6 nM, while there was no effect on growth for the low Her2/neu expressing cells (FIG. 7B). Incubation of cells simultaneously with the two antibodies shows only a minor increase in inhibition of proliferation (data not shown).

In another set of experiments the antiproliferative effect of Fab63 was tested in the presence of HRG-β1, the ligand of Her3 receptor. Fab63 significantly inhibited cell proliferation both in the high Her2/neu MDA-MB-453 and low Her2/neu MCF7 cells, up to 34.5% (p<0.05) and 21% (p<0.05) respectively, compared to non Fab treated cells (FIG. 8). Herceptin did not induce any significant inhibition on cell growth (p>0.1) to both cell lines in the presence of HRG-β1. As it was expected, in the absence of the ligand, both Fab63 and Herceptin inhibited cell proliferation in the high Her2/neu expressing cells, while there was low inhibition observed in the MCF7 cell line.

These data show that Fab63 has significant antiproliferative effects in SKBR3 and MDA-MB-453 cancer cells where ligand-independent mechanisms dominate signal induction, while is also able to negatively affect cell growth in the presence of HRG-β1 growth factor in both MDA-MB-453 and the low expressing Her2/neu MCF7 cell line, implicating involvement in ligand-dependent proliferation.

### Discussion

This study describes the successful isolation of a fully human antibody fragment, Fab63, against the extracellular domain of Her2/neu oncoprotein that was soluble expressed in yeast P. pastoris. The Fab63 couples its property to rapidly internalize in cell cytoplasm with strong antiproliferative effects in high expressing Her2/neu cancer cells.

A characteristic event in metastatic breast cancers is the elevated levels of Her2/neu ECD in patient sera (Molina et al., 2002; Wu, 2002) Furthermore, the presence of Her2/neu antibodies and their correlation with Her2/neu positive cancer, implies that immunity of Her2/neu develops as a result of exposure of patients to Her2/neu protein expressed by their own cancer (Disis et al., 1997). Several groups have isolated mouse and rat mAbs against the Her2/neu ECD (Hudziak R M., 1989; Harwerth IM., 1993; Kita Y., 1996), which present immunotherapeutic properties, such as antiproliferative effect on tumor cells. However, their nonhuman origin has limited use as the repeated treatment produce human anti-mouse antibodies (the HAMA response). The ability to manipulate rodent antibodies into more human variants allowed the production of therapeutic agents with reduced immunogenic potential (Carter P., 1992). Herceptin and Pertuzumab, the humanized variants of two mouse mAbs, are introduced as therapeutic agents for targeting high and/or low expressing Her2/neu cancers, respectively (Franklin et al., 2004; Slamon et al., 2001). Recently, by using the phage display technology, fully human scFvs or Fabs are selected from large combinatorial libraries of human antibody fragments (Burton and Barbas, 1994; Marks et al., 1991). Moreover, the in vivo antibody responses to tumour-associated antigens may be exploited in vitro for the production of tumour-specific recombinant antibodies. According to this, human Fab combinatorial library from the lymph nodes of an advanced breast cancer patient produced a rich source of anti-Her2/neu antibody fragments (Clark et al., 1997). In order to isolate an agent with antitumor activity, human scFv combinatorial phage display libraries were panned on recombinant ECD expressed in CHO cells, but without succeeded isolation (Schier R., 1995; Sheets M D., 1998). Recently, a phage scFv (Erbicin) was selected by panning the phage library on live Her2/neu overexpressing cells, using a subtractive selection strategy based on the use of two combinations of Her2/neu-positive and -negative cell lines (De Lorenzo et al., 2002). This scFv fragment in order to have a more stable conformation was reconstructed as a compact reduced version of an IgG that retains the antiproliferative properties of the original antibody fragment (De Lorenzo et al., 2004).

In this study, we used the extracellular domain of the Her2/neu antigen expressed in the methylotrophic yeast Pichia pastoris, in order to isolate anti-Her2/neu Fab fragments from an immune phage display library. The heterologous expression system of Pichia pastoris has an efficient secretory pathway that allows the recombinant protein to be secreted into the medium at high yield. Moreover, it allows post-translation modifications including glycosylation and is less prone to hyperglycosylation than Saccharomyces cerevisiae (Cregg et al., 2000; Gellissen, 2000; Hollenberg and Gellissen, 1997; Vedvick et al., 1991). Different conformation dependent anti-Her2/neu mAbs can bind to the recombinant ECD fragment suggesting that the ECD resembles the native protein. This fragment was also used to efficiently select phage antibodies recognizing specifically the Her2/neu antigen, from a Fab combinatorial library constructed from infiltrated B lymphocytes of a patient with Her2/neu overexpressing tumour. As was noted previously, T-helper, cytotoxic and antibody response have been identified in patients whose tumors overexpress Her2/neu (Disis et al., 1994) and furthermore several peptide sequences within Her2/neu as well as DNA vaccines were identified that presented anti-tumor immunity in mouse (Yoshino et al., 1994; Peoples et al., 1995; Piechocki et al., 2001).

The selected Fab fragments from the immune library were examined for cross-reactivity by ELISA and shown to be negative against a panel of irrelevant antigens, including bovine serum albumin (BSA) and the antigens human serum albumin (HSA) and α1-ECD of AchR expressed in Pichia pastoris. Since our selection was based on a purified recombinant fragment of the Her2/neu antigen it was essential to confirm that Fab63 is able to recognize the native Her2/neu protein and represent the in vivo conditions. Fab63 recognized the soluble Her2/neu receptor and specifically bound to the high Her2/neu expressing SKBR3 cell line and not to the HeLa cells as shown by immunofluorescence analysis.

In a primal attempt to direct Fab63 binding towards a proximate site along the ˜630 residue domain, we used a number of available anti-Her2 antibodies in competition assays with Her2/neu receptor. There was a strong competition effect only between Fab63 and Herceptin suggesting a possible relationship in the binding site of the two antibodies. Herceptin Fab-Her2/neu ECD complex has been crystalographically resolved, and revealed antibody binding at the C-end of the ECD IV domain (Cho et al., 2003). A considerable credit of the anti-tumor properties of Herceptin was given to this juxtamembrane binding site, which inhibits cleavage of ECD in breast cancer cells and blocks Her2/neu self-association and constitutive kinase activation (Molina et al., 2001).

What differentiates Fab63 from Herceptin and other antibodies is that its antiproliferative effects are combined with its ability to strongly and rapidly internalize in target cells. Previous studies have shown that neither monovalent nor bivalent scFv forms of the mAb4D5, the original mouse version of Herceptin, have any significant growth altering effects on ErbB2 overepressing cells (Neve R M. et al., 2001). Furthermore, there are no reports for anti-Her2/neu Fab monovalent fragments pairing these two dynamics although internalized Fabs without anti-tumor properties have been described before (Poul et al., 2000). The previous described phage scFv or the respective soluble scFv (Erbicin) with anti-tumor properties, have been internalized in SKBR3 cells within 16 hours of incubation at 37° C. (De Lorenzo et al., 2002). In contrast, Fab63 was rapidly internalized within 30 minutes of incubation with the same cells and exerts its maximum efficiency of internalization within 2 hours at 37° C. In these experiments, Herceptin stained only the membrane of the cells at the corresponding time intervals, although a recent study shows detection of Trastuzumab (Herceptin) internalization after 3 h at 37° C. (Austin et al., 2004). Moreover, Fab63 has the ability to inhibit cell growth in the presence of Her3 ligand, HRG-β1, in high and low Her2/neu expressing cells, whereas Herceptin could not induce a similar effect. The mechanisms of Fab63 cell growth inhibition is under investigation.

In conclusion, a new fully human Fab fragment was isolated against the extracellular domain of Her2/neu protein expressed in yeast P. pastoris, which has strong antiproliferative effects and internalization properties.

### REFERENCES

Agus D. B., Akita R. W., Fox W. D., Lewis G. D., Higgins B., Pisacane P. I., Lofgren J. A., Tindell C., Evans D. P., Maiese K., Scher H. I. and Sliwkowski M. X. (2002) Targeting ligand-activated ErbB2 signaling inhibits breast and prostate tumor growth. Cancer Cell 2, 127-37.
Akiyama T., Kadooka T., Ogawara H. and Sakakibara S. (1986) Characterization of the epidermal growth factor receptor and the erbB oncogene product by site-specific antibodies. Arch Biochem Biophys 245, 531-6.
Austin C. D., De Maziere A. M., Pisacane P. I., van Dijk S. M., Eigenbrot C., Sliwkowski M. X., Klumperman J. and Scheller R. H. (2004) Endocytosis and sorting of ErbB2 and the site of action of cancer therapeutics trastuzumab and geldanamycin. Mol Biol Cell 15, 5268-82.
Barbas C. F., 3rd, Kang A. S., Lerner R. A. and Benkovic S. J. (1991) Assembly of combinatorial antibody libraries on phage surfaces: the gene III site. Proc Natl Acad Sci USA 88, 7978-82.
Brekke O. H. and Sandlie I. (2003) Therapeutic antibodies for human diseases at the dawn of the twenty-first century. Nat Rev Drug Discov 2, 52-62.
Burgess A. W., Cho H. S., Eigenbrot C., Ferguson K. M., Garrett T. P., Leahy D. J., Lemmon M. A., Sliwkowski M. X., Ward C. W. and Yokoyama S. (2003) An open-and-shut case? Recent insights into the activation of EGF/ErbB receptors. Mol Cell 12, 541-52.
Burton D. R. (1991) Human and mouse monoclonal antibodies by repertoire cloning. Trends Biotechnol 9, 169-75.
Burton D. R. and Barbas C. F., 3rd. (1994) Human antibodies from combinatorial libraries. Adv Immunol 57, 191-280.
Camirand A., Lu Y. and Pollak M. (2002) Co-targeting HER2/ErbB2 and insulin-like growth factor-1 receptors causes synergistic inhibition of growth in HER2-overexpressing breast cancer cells. Med Sci Monit 8, BR521-6.
Carter P. (2001) Improving the efficacy of antibody-based cancer therapies. Nat Rev Cancer 1, 118-29.
Cho H. S., Mason K., Ramyar K. X., Stanley A. M., Gabelli S. B., Denney D. W., Jr. and Leahy D. J. (2003) Structure of the extracellular region of HER2 alone and in complex with the Herceptin Fab. Nature 421, 756-60.
Chomczynski P. and Sacchi N. (1987) Single-step method of RNA isolation by acid guanidinium thiocyanate-phenol-chloroform extraction. Anal Biochem 162, 156-9.
Christianson T. A., Doherty J. K., Lin Y. J., Ramsey E. E., Holmes R., Keenan E. J. and Clinton G. M. (1998) NH2-terminally truncated HER-2/neu protein: relationship with shedding of the extracellular domain and with prognostic factors in breast cancer. Cancer Res 58, 5123-9.
Citri A., Skaria K. B. and Yarden Y. (2003) The deaf and the dumb: the biology of ErbB-2 and ErbB-3. Exp Cell Res 284, 54-65.
Clark M. A., Hawkins N. J., Papaioannou A., Fiddes R. J. and Ward R. L. (1997) Isolation of human anti-c-erbB-2 Fabs from a lymph node-derived phage display library. Clin Exp Immunol 109, 166-74.
Cregg J. M., Cereghino J. L., Shi J. and Higgins D. R. (2000) Recombinant protein expression in Pichia pastoris. Mol Biotechnol 16, 23-52.
De Lorenzo C., Palmer D. B., Piccoli R., Ritter M. A. and D'Alessio G. (2002) A new human antitumor immunoreagent specific for ErbB2. Clin Cancer Res 8, 1710-9.
De Lorenzo C., Tedesco A., Terrazzano G., Cozzolino R., Laccetti P., Piccoli R. and D'Alessio G. (2004) A human, compact, fully functional anti-ErbB2 antibody as a novel antitumour agent. Br J Cancer 91, 1200-4.
Disis M L, Smith J W, Murphy A E, Chen W, Cheever M. A. (1994) In vitro generation of human cytolytic T-cells specific for peptides derived from the HER-2/neu protooncogene protein. Cancer Res. 54, 1071-6.
Disis M. L., Pupa S. M., Gralow J. R., Dittadi R., Menard S, and Cheever M. A. (1997) High-titer HER-2/neu protein-specific antibody can be detected in patients with early-stage breast cancer. J Clin Oncol 15, 3363-7.
Fominaya J. and Wels W. (1996) Target cell-specific DNA transfer mediated by a chimeric multidomain protein. Novel non-viral gene delivery system. J Biol Chem 271, 10560-8.
Fostieri E., Tzartos S J, Berrih-Aknin S, Beeson D, Mamalaki A. (2005) Isolation of potent human Fab fragments against a novel highly immunogenic region on human muscle acetylcholine receptor which protect the receptor from myasthenic autoantibodies. Eur J Immunol. (in press)
Franklin M. C., Carey K. D., Vajdos F. F., Leahy D. J., de Vos A. M. and Sliwkowski M. X. (2004) Insights into ErbB signaling from the structure of the ErbB2-pertuzumab complex. Cancer Cell 5, 317-28.
Garrett T. P., McKern N. M., Lou M., Elleman T. C., Adams T. E., Lovrecz G. O., Kofler M., Jorissen R. N., Nice E. C., Burgess A. W. and Ward C. W. (2003) The crystal structure of a truncated ErbB2 ectodomain reveals an active conformation, poised to interact with other ErbB receptors. Mol Cell 11, 495-505.
Gellissen G. (2000) Heterologous protein production in methylotrophic yeasts. Appl Microbiol Biotechnol 54, 741-50.
Graus Y. F., de Baets M. H., van Breda Vriesman P. J. and Burton D. R. (1997) Anti-acetylcholine receptor Fab fragments isolated from thymus-derived phage display libraries from myasthenia gravis patients reflect predominant specificities in serum and block the action of pathogenic serum antibodies. Immunol Lett 57, 59-62.
Harari D. and Yarden Y. (2000) Molecular mechanisms underlying ErbB2/HER2 action in breast cancer. Oncogene 19, 6102-14.
Harris M. (2004) Monoclonal antibodies as therapeutic agents for cancer. Lancet Oncol 5, 292-302.
Holbro T., Beerli R. R., Maurer F., Koziczak M., Barbas C. F., 3rd and Hynes N. E. (2003) The ErbB2/ErbB3 heterodimer functions as an oncogenic unit: ErbB2 requires ErbB3 to drive breast tumor cell proliferation. Proc Natl Acad Sci U S A 100, 8933-8.
Hollenberg C. P. and Gellissen G. (1997) Production of recombinant proteins by methylotrophic yeasts. Curr Opin Biotechnol 8, 554-60.
Kabat E. A. T. T. W., H. M. Perry, K. S. Gottesman, and C. Foeller. Sequences of Proteins of Immunological Interest, Bethesda, Md. 1991.
Koeppen H. K., Wright B. D., Burt A. D., Quirke P., McNicol A. M., Dybdal N. O., Sliwkowski M. X. and Hillan K. J. (2001) Overexpression of HER2/neu in solid tumours: an immunohistochemical survey. Histopathology 38, 96-104.
Lacroix M. and Leclercq G. (2004) Relevance of breast cancer cell lines as models for breast tumours: an update. Breast Cancer Res Treat 83, 249-89.
Lu Y., Zi X., Zhao Y., Mascarenhas D. and Pollak M. (2001) Insulin-like growth factor-I receptor signaling and resistance to trastuzumab (Herceptin). J Natl Cancer Inst 93, 1852-7.
Marks J. D., Hoogenboom H. R., Bonnert T. P., McCafferty J., Griffiths A. D. and Winter G. (1991) By-passing immunization. Human antibodies from V-gene libraries displayed on phage. J Mol Biol 222, 581-97.
Marmor M. D., Skaria K. B. and Yarden Y. (2004) Signal transduction and oncogenesis by ErbB/HER receptors. Int J Radiat Oncol Biol Phys 58, 903-13.
Mellinghoff I. K., Vivanco I., Kwon A., Tran C., Wongvipat J. and Sawyers C. L. (2004) HER2/neu kinase-dependent modulation of androgen receptor function through effects on DNA binding and stability. Cancer Cell 6, 517-27.
Molina M. A., Codony-Servat J., Albanell J., Rojo F., Arribas J. and Baselga J. (2001) Trastuzumab (herceptin), a humanized anti-Her2 receptor monoclonal antibody, inhibits basal and activated Her2 ectodomain cleavage in breast cancer cells. Cancer Res 61, 4744-9.
Molina M. A., Saez R., Ramsey E. E., Garcia-Barchino M. J., Rojo F., Evans A. J., Albanell J., Keenan E. J., Lluch A., Garcia-Conde J., Baselga J. and Clinton G. M. (2002) NH(2)-terminal truncated HER-2 protein but not full-length receptor is associated with nodal metastasis in human breast cancer. Clin Cancer Res 8, 347-53.
Nahta R., Hung M. C. and Esteva F. J. (2004) The HER-2-targeting antibodies trastuzumab and pertuzumab synergistically inhibit the survival of breast cancer cells. Cancer Res 64, 2343-6.
Park J. W., Hong K., Kirpotin D. B., Colbern G., Shalaby R., Baselga J., Shao Y., Nielsen U. B., Marks J. D., Moore D., Papahadjopoulos D. and Benz C. C. (2002) Anti-HER2 immunoliposomes: enhanced efficacy attributable to targeted delivery. Clin Cancer Res 8, 1172-81.
Peoples G E, Smith R C, Linehan D C, Yoshino I, Goedegebuure P S, Eberlein T J. (1995) Shared T cell epitopes in epithelial tumors. Cell Immunol. 164, 279-86.
Piechocki M P, Pilon S A, Wei W Z. (2001) Complementary antitumor immunity induced by plasmid DNA encoding secreted and cytoplasmic human ErbB-2. J Inmmunol. 167, 3367-74.
Poul M. A., Becerril B., Nielsen U. B., Morisson P. and Marks J. D. (2000) Selection of tumor-specific internalizing human antibodies from phage libraries. J Mol Biol 301, 1149-61.
Press M. F., Pike M. C., Chazin V. R., Hung G., Udove J. A., Markowicz M., Danyluk J., Godolphin W., Sliwkowski M., Akita R. and et al. (1993) Her-2/neu expression in node-negative breast cancer: direct tissue quantitation by computerized image analysis and association of overexpression with increased risk of recurrent disease. Cancer Res 53, 4960-70.
Psaridi-Linardaki L, Mamalaki A, Remoundos M, Tzartos S J. (2002) Expression of soluble ligand- and antibody-binding extracellular domain of human muscle acetylcholine receptor alpha subunit in yeast Pichia pastoris. Role of glycosylation in alpha-bungarotoxin binding. J Biol. Chem. 277, 26980-6.
Ross J. S, and Fletcher J. A. (1998) The HER-2/neu oncogene in breast cancer: prognostic factor, predictive factor, and target for therapy. Stem Cells 16, 413-28.
Ross J. S., Fletcher J. A., Linette G. P., Stec J., Clark E., Ayers M., Symmans W. F., Pusztai L. and Bloom K. J. (2003) The Her-2/neu gene and protein in breast cancer 2003: biomarker and target of therapy. Oncologist 8, 307-25.
Rubin I. and Yarden Y. (2001) The basic biology of HER2. Ann Oncol 12 Suppl 1, S3-8.
Slamon D. J. (1987) Proto-oncogenes and human cancers. N Engl J Med 317, 955-7.
Slamon D. J., Leyland-Jones B., Shak S., Fuchs H., Paton V., Bajamonde A., Fleming T., Eiermann W., Wolter J., Pegram M., Baselga J. and Norton L. (2001) Use of chemotherapy plus a monoclonal antibody against HER2 for metastatic breast cancer that overexpresses HER2. N Engl J Med 344, 783-92.
Tzahar E., Pinkas-Kramarski R., Moyer J. D., Klapper L. N., Alroy I., Levkowitz G., Shelly M., Henis S., Eisenstein M., Ratzkin B. J., Sela M., Andrews G. C. and Yarden Y. (1997) Bivalence of EGF-like ligands drives the ErbB signaling network. Embo J 16, 4938-50.
Vedvick T., Buckholz R. G., Engel M., Urcan M., Kinney J., Provow S., Siegel R. S, and Thill G. P. (1991) High-level secretion of biologically active aprotinin from the yeast Pichia pastoris. J Ind Microbiol 7, 197-201.
Wang J. N., Feng J. N., Yu M., Xu M., Shi M., Zhou T., Yu X. D., Shen B. F. and Guo N. (2004) Structural analysis of the epitopes on erbB2 interacted with inhibitory or non-inhibitory monoclonal antibodies. Mol Immunol 40, 963-9.
Wang L., Liu B., Schmidt M., Lu Y., Wels W. and Fan Z. (2001) Antitumor effect of an HER2-specific antibody-toxin fusion protein on human prostate cancer cells. Prostate 47, 21-8.
Williamson R. A., Burioni R., Sanna P. P., Partridge L. J., Barbas C. F., 3rd and Burton D. R. (1993) Human monoclonal antibodies against a plethora of viral pathogens from single combinatorial libraries. Proc Natl Acad Sci USA 90, 4141-5.
Wu J. T. (2002) C-erbB2 oncoprotein and its soluble ectodomain: a new potential tumor marker for prognosis early detection and monitoring patients undergoing Herceptin treatment. Clin Chim Acta 322, 11-9.
Yarden Y. and Sliwkowski M. X. (2001) Untangling the ErbB signalling network. Nat Rev Mol Cell Biol 2, 127-37.
Yoshino I, Goedegebuure P S, Peoples G E, Parikh A S, DiMaio J M, Lyerly H K, Gazdar A F, Eberlein T J. (1994) HER2/neu-derived peptides are shared antigens among human non-small cell lung cancer and ovarian cancer. Cancer Res. 54, 3387-90.
Zabrecky J. R., Lam T., McKenzie S. J. and Carney W. (1991) The extracellular domain of p185/neu is released from the surface of human breast carcinoma cells, SK-BR-3. J Biol Chem 266, 1716-20.

All publications mentioned in the above specification, and references cited in said publications, are herein incorporated by reference. Various modifications and variations of the described methods and system of the present invention will be apparent to those skilled in the art without departing from the scope and spirit of the present invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. An isolated antibody or a fragment, variant or derivative thereof, capable of specifically binding to Her2/neu comprising a heavy chain variable domain wherein said heavy chain variable domain comprises an amino acid sequence as set out in at least one of SEQ ID NOs: 1, 2 or 3 or a sequence having at least 75% identity (homology) thereto or a functional fragment thereof.

2. An isolated antibody as claimed in claim 1 further comprising a light chain variable domain wherein said light chain variable domain comprises an amino acid sequence as set out in at least one of SEQ ID NOs: 4, 5 or 6 or a sequence having at least 75% identity (homology) thereto or a functional fragment thereof.

3. An isolated antibody as claimed in claim 1 comprising at least one of the amino acid sequences shown in any of SEQ ID NOs: 1 to 6 or a sequence having at least 75% identity (homology) thereto or a functional fragment thereof.

4. An isolated antibody as claimed in claim 1 comprising a combination of at least two of SEQ ID NOs: 1 to 6.

5. An isolated antibody as claimed in claim 1 comprising a heavy chain variable domain comprising a combination of all of SEQ ID NOs: 1 to 3.

6. An isolated antibody as claimed in claim 1 comprising a light chain variable domain comprising a combination of all of SEQ ID NOs: 4 to 6.

7. An isolated antibody as claimed in claim 5 comprising a heavy chain variable domain having an amino acid sequence as set out in SEQ ID NO: 7.

8. An isolated antibody as claimed claim 6 comprising a light chain variable domain having an amino acid sequence as set out in SEQ ID NO: 8.

9. An isolated antibody as claimed in claim 1 comprising a heavy chain variable domain having an amino acid sequence as set out in SEQ ID NO: 7 in combination with a light chain variable domain having an amino acid sequence as set out in SEQ ID NO: 8.

10. An isolated antibody as claimed in claim 1 wherein said antibody is capable of rapid internalisation.

11. An isolated antibody as claimed in claim 1 wherein said antibody is able to exert an antiproliferative effect when provided to a cell expressing Her2/neu.

12. An isolated antibody as claimed in claim 1 wherein said antibody is an Fv fragment comprising a CDR as set out in any of SEQ ID NOs: 1 to 6 and framework regions.

13. An isolated antibody as claimed in claim 1 wherein said antibody is a Fab fragment.

14. An isolated antibody as claimed in claim 1 wherein said antibody has been modified so as to increase affinity, stability and/or half-life.

15. An isolated antibody as claimed in claim 14 wherein said modified antibody is a whole antibody or antibody mutated by chain shuffling technology.

16. An isolated antibody as claimed in claim 1 conjugated to a second molecule wherein the second molecule is selected from the group consisting of a cytotoxic drug, cytostatic drug, immunotoxin, immunoliposome, DNA molecule.

17. An isolated polypeptide having an amino acid sequence as set out in any of SEQ ID NOs: 1 to 8 or a sequence having at least 75% identity (homology) thereto or a functional fragment thereof.

18. An isolated nucleic acid molecule encoding any one or more antibody molecules or polypeptides as claimed in claim 1 or a homologue thereof.

19. An isolated nucleic acid molecule as claimed in claim 18 which encodes an antibody comprising the amino acid sequence as shown in any of SEQ ID NOs: 1 to 8 or a sequence having at least 75% identity (homology) thereto or an effective fragment thereof.

20. An isolated nucleic acid molecule as claimed in claim 18 wherein said nucleic acid molecule comprises a sequence as set out in any of SEQ ID NOs: 9 to 16.

21. A plasmid or vector system comprising a nucleic acid encoding an antibody as claimed in claim 1 or a homologue or derivative thereof.

22. A plasmid or vector system comprising a nucleic acid as claimed in claim 18.

23. A host cell transformed or transfected with a nucleic acid molecule as claimed in claim 8.

24. A composition comprising an antibody molecule as claimed in claim 1, and a pharmaceutically acceptable carrier, diluent or excipient.

25. A method for the treatment of Her2/neu expressing cancer in a patient comprising administering to the patient in need of such treatment a therapeutically effective amount of an antibody molecule as claimed in claim 1.

26. Use of an antibody molecule as claimed in claim 1, in the preparation of a medicament for use in the treatment of cancers expressing Her2.

27. A method for diagnosing a cancer expressing Her2/neu comprising contacting a sample with an antibody as claimed in claim 1.

28. Use of an antibody as claimed in claim 1 in diagnosis.


SEQUENCE LISTING (16 sequences)

SEQ ID NO: 1 - protein, 6 aa, Artificial
  Heavy chain CDR
DSNWWG

SEQ ID NO: 2 - protein, 16 aa, Artificial
  Heavy chain CDR
EIYHSGTTNYNPSLKS

SEQ ID NO: 3 - protein, 12 aa, Artificial
  Heavy chain CDR
EDCSYGSCYFDY

SEQ ID NO: 4 - protein, 11 aa, Artificial
  Light chain CDR
RASQSISAALN

SEQ ID NO: 5 - protein, 7 aa, Artificial
  Light chain CDR
DVSTLQS

SEQ ID NO: 6 - protein, 9 aa, Artificial
  Light chain CDR
QQSYRVPLT

SEQ ID NO: 7 - protein, 121 aa, Artificial
  Heavy chain sequence
EVQLLEWGPGLVKPSGTLSLTCAVSGGSIRDSNWWGWVRQPPGKGLEWIGEIYHSGTTNYNPSLKSRVTISVDKSKNQFSLNLSSVTAADTAVYYCAREDCSYGSCYFDYWGQGTLVTVSS

SEQ ID NO: 8 - protein, 107 aa, Artificial
  Light chain sequence
ELTLTQSPSSLSASLGDRVTIRCRASQSISAALNWYQQKPGKAPKLLIHDVSTLQSGVPARFSGGGSETDFTLTISSLQPEDFATYYCQQSYRVPLTFGGGTRLEIK

SEQ ID NO: 9 - DNA, 18 bp, Artificial
  Heavy chain CDR
gatagtaattggtggggt

SEQ ID NO: 10 - DNA, 48 bp, Artificial
  Heavy chain CDR
gaaatctatcatagtggaaccaccaactacaacccgtccctcaagagt

SEQ ID NO: 11 - DNA, 36 bp, Artificial
  Heavy chain CDR
gaggattgcagttatggtagctgctactttgactac

SEQ ID NO: 12 - DNA, 33 bp, Artificial
  Light chain CDR
cgggcaagtcagagcattagcgccgctttaaat

SEQ ID NO: 13 - DNA, 21 bp, Artificial
  Light chain CDR
gatgtgtccactttgcaaagt

SEQ ID NO: 14 - DNA, 27 bp, Artificial
  Light chain CDR
caacagagttaccgtgttccgctcact

SEQ ID NO: 15 - DNA, 362 bp, Artificial
  heavy chain sequence
gaggtgcagctgctcgagtggggcccaggactggtgaagccttcggggaccctgtccctcacctgcgctgtctctggtggctccatcagggatagtaattggtggggttgggtccgccagcccccagggaaggggctggagtggattggggaaatctatctagtggaaccaccaactacaacccgtccctcaagagtcgagtcaccatatcagtagacaagtccaagaaccagttctccctgaacctgagttctgtgaccgccgcggacacggccgtttattactgtgcgagagaggattgcagttatggtagctgctactttgactactggggccagggaaccctggtcaccgtctcctca

SEQ ID NO: 16 - DNA, 321 bp, Artificial
  light chain sequence
gagctcacactcacgcagtctccatcctccctgtctgcatctttaggggacagagtcaccatcaggtgccgggcaagtcagagcattagcgccgctttaaattggtatcagcagaaaccagggaaagcccctaagctcctgattcatgatgtgtccactttgcaaagtggggtccctgcaaggttcagtggcggggggtctgagacagatttcactctcaccatcagcagtctgcagcctgaagattttgcaacttactactgtcaacagagttaccgtgttccgctcactttcggcggagggacccggctagagatcaag